(19) United States
(12) Patent Application Publication
(10) Pub. No.: US 2023/0035859 A1
(43) Pub. Date: Feb. 2, 2023

(54) COMPOSITIONS AND METHODS FOR EPITOPE SCANNING

(71) Applicant: The Brigham and Women's Hospital, Inc., Boston, MA (US)

(72) Inventors: Peter M. Bruno, Brookline, MA (US); Stephen J. Elledge, Brookline, MA (US)

(21) Appl. No.: 17/780,407

(22) PCT Filed: Dec. 2, 2020
(86) PCT No.: PCT/US2020/062912

§ 371 Date: May 26, 2022

Related U.S. Application Data
(60) Provisional application No. 62/942,428, filed on Dec. 2, 2019.

(51) Int. Cl. C12N 15/113; C07K 14/74; C07K 14/005; C12N 5/00; C12N 15/62; C12N 15/86; C12N 15/10

(52) U.S. Cl. CPC C12N 15/1138; C07K 14/70539; C07K 14/005; C12N 5/00; C12N 15/625; C12N 15/86; C12N 15/1037; C12N 2740/15043

(57) ABSTRACT

Described herein are methods for identification of peptides that bind MHC-I molecules from within a starting pool of candidate epitope peptides, using a cell-based genetic immunopeptidomic screen.

### CLAIM OF PRIORITY

This application claims the benefit of U.S. Provisional Application Ser. No. 62/942,428, filed on Dec. 2, 2019. The entire contents of the foregoing are incorporated herein by reference.

### FEDERALLY SPONSORED RESEARCH OR DEVELOPMENT

This invention was made with Government support under Grant No. BC171184 awarded by the Department of Defense. The Government has certain rights in the invention.

### TECHNICAL FIELD

Described herein are methods for identification of peptides that bind MIHC-I molecules from within a starting pool of candidate epitope peptides, using a cell-based genetic immunopeptidomic screen, and for generating cells that display only one or a selected set of peptide:MHC complexes on the cell surface.

### BACKGROUND

The immune system samples the internal protein environment of all cells via the human leukocyte antigen (HLA) Class I (HLA-I) presentation system (HLA is the major histocompatibility complex (MIHC) in humans). Non-self or altered peptides displayed by HLA-I can elicit an immune response against those peptides through the activation of CD8+ Cytotoxic T cells. In some cases, nonmutant self-peptides displayed by HLA-I can elicit a response leading to autoimmunity. In a normal cell, proteins are digested by the proteosome in the cytosol into peptides of varying length. Those peptides, typically ranging from about 7 to 20 amino acids, are imported into the endoplasmic reticulum (ER) by a complex of two proteins, Transporter 1, ATP Binding Cassette Subfamily B Member 1 (TAP1) and TAP2. In the ER, two N-terminal peptidases, endoplasmic reticulum aminopeptidase 1 (ERAP1) and ERAP2, trim the peptides down, including to around 7-13 or 8-9 amino acids. Finally, the HLA-I proteins, HLA-A, -B and -C, sample peptides, generally in the range of 7-13 or 8-12 amino acids, and once bound sufficiently tightly, traffic to and present the peptides on the cell surface.

The presentation of intracellular peptides on the cell surface allows surveilling cytotoxic CD8⁺ T cells to identify pathogen-infected or malignant cells¹. A better understanding of the rules governing peptide binding by MIHC-I molecules would facilitate the development of more effective vaccines and other immune-based therapies, but this task is complicated by the diverse array of MIHC-I molecules (HLA-A, -B, -C, -E and -G) expressed in human cells and their highly polymorphic nature across the human population³. Mass spectrometry (MS) is currently the leading method for identifying MIHC-I ligands, with large-scale experiments capable of identifying roughly a thousand peptides eluted from any given HLA allele⁴. One key limitation, however, is that MS-based approaches must inevitably sample peptides derived from the entire cellular proteome, and cannot be readily adapted to permit the targeted evaluation of T cell epitopes generated from a particular pathogen or neo-antigens presented by a particular tumour.

### SUMMARY

Described herein are methods and compositions for rapid empirical determination of MIHC-I binding for large pools of peptides, leveraging inexpensive DNA oligonucleotide synthesis to generate pre-defined libraries for targeted immunopeptidomics. The system can be used for querying individual peptides for MIHC-I binding, and has a number of applications.

Provided herein are isolated cells, wherein the cell has been engineered or modified to lack expression of two, three, four, or more, preferably all, of: human leukocyte antigen A (HLA-A); HLA-B; HLA-C; Transporter 1, ATP Binding Cassette Subfamily B Member 1 (TAP1); TAP2; endoplasmic reticulum aminopeptidase 1 (ERAP1); ERAP2; and histocompatibility minor 13 (HM13), and wherein the cell expresses a single HLA allele.

In some embodiments, the cell lacks expression of TAP1; TAP2; ERAP1; ERAP2; and HM13; and lacks expression of at least two of HLA-A; HLA-B; and HLA-C.

In some embodiments, the cell lacks expression of TAP1; TAP2; ERAP1; ERAP2; HM13; HLA-A; HLA-B; HLA-C, and expresses an exogenous HLA-I allele.

In some embodiments, the cell is a mammalian cell, preferably a human cell. Non mammalian cells can also be used, e.g., insect or avian cells; any cell type that can be engineered to place MHC, B2M peptide complexes on the surface of cells can be used.

In some embodiments, the cell further comprises (i) a nucleic acid comprising one or more sequences encoding candidate epitope peptides, e.g., 8-12mer, 9-mer, or longer, candidate epitope peptides, linked to a signal peptide that is preferably at least 16, 17, or 18 amino acids long and directs the peptide to the endoplasmic reticulum (ER), and a promoter that drives expression of the candidate epitope peptide linked to a signal peptide; or (ii) candidate epitope peptides, e.g., 8-12mer, 9-mer, or longer candidate epitope peptides linked to a signal peptide that is preferably at least 16, 17, or 18 amino acids long and directs the peptide to the ER. In some embodiments, the signal peptide comprises a MMTV gp70 signal peptide.

In some embodiments, the cell expresses the candidate epitope peptides linked to a signal peptide, and the candidate epitope peptides are trafficked to the ER.

Also provided herein are methods for identifying an MHC-I binding peptide. In some embodiments, the methods include providing a sample comprising the cells described herein that express a selected MHC-I allele; expressing in the cells a plurality of different candidate epitope peptides, such that each cell expresses a single selected candidate epitope peptide or plurality of candidate epitope peptides; isolating cells that have cell surface expression of the MHC-I allele; and identifying candidate epitope peptides in the cells that have cell surface expression of the MHC-I allele, thereby identifying peptides that bind to the MHC-I allele.

In some embodiments, expressing in the cells a plurality of different candidate epitope peptides comprises contacting the cells with a plurality of nucleic acids each comprising one or more sequences encoding 8-12mer, preferably 9-mer, candidate epitope peptides linked to a signal peptide that is at least 16, 17, or 18 amino acids long and directs the peptide to the endoplasmic reticulum (ER), and a promoter that drives expression of the candidate epitope peptide linked to the signal peptide, under conditions sufficient for the cells to express the peptides, preferably wherein the signal peptide comprises a MMTV gp70 signal peptide.

In some embodiments, the nucleic acids comprise expression vectors. In some embodiments, the expression vectors are viral expression vectors or plasmids. In some embodiments, the viral expression vectors are retroviral, preferably lentiviral, vectors.

In some embodiments, each cell expresses one to 100, e.g., 2, 3, 4, 5, 6, 7, 8, 9, 10, 12, 20, 24, 30, 36 or more, e.g., up to 50 or 100, different candidate epitope peptides, but does not express any other peptides in the ER.

In some embodiments, the plurality of different candidate epitope peptides comprise random sequences.

In some embodiments, the plurality of different candidate epitope peptides comprise sequences derived from a pathogen, preferably a viral, bacterial, parasitic, or fungal pathogen, or from a cancer antigen.

In some embodiments, the plurality of different candidate epitope peptides comprise sequences from an autoantigen or potential autoantigen.

In some embodiments, the plurality of different candidate epitope peptides comprise an entire peptidome (peptides representing some or all of the genome of an organism).

In some embodiments, the methods include expressing at least 100; 1,000; 10,000; 100,000; 200,000; 250,000; 300,000; or more different candidate epitope peptides.

In some embodiments, isolating cells that have cell surface expression of an MHC allele comprises using fluorescence-activated cell sorting (FACS) or magnetic-activated cell sorting (MACS).

In some embodiments, identifying candidate epitope peptides comprises determining sequences encoding the peptides expressed in the cells that have cell surface expression of an MHC allele.

In some embodiments, the sequences encoding the peptides are determined by sequencing.

Additionally, provided herein are methods for isolating a cell for use in generating an immune response to an epitope in a subject. The methods can include providing a sample comprising the cells of claims 1 to 4 that express a selected MHC-I allele; expressing in the cells a plurality of different candidate epitope peptides linked to a signal peptide that is preferably at least 16, 17, or 18 amino acids long and directs the peptide to the endoplasmic reticulum (ER), such that each cell expresses a single selected candidate epitope peptide or plurality of candidate epitope peptides; and isolating cells that have cell surface expression of the MHC-I allele. In some embodiments, the plurality of different candidate epitope peptides comprise sequences derived from a pathogen, preferably a viral, bacterial, parasitic, or fungal pathogen, or from a cancer antigen.

Also provided herein are methods for stimulating T cells, or providing populations of stimulated/activated T cells. The methods can include providing a sample comprising the cells of claims 1 to 4 that express a selected MHC-I allele; expressing in the cells one or more specific epitope peptide linked to a signal peptide that is preferably at least 16, 17, or 18 amino acids long and directs the peptide to the endoplasmic reticulum (ER), such that each cell expresses a single specific epitope peptide or plurality of specific epitope peptides; incubating the cells in the presence of T cells in culture under conditions that allow activation of the T cells; and isolating activated T cells from the culture. These methods can be used to stimulate T cells in vitro to evolve T cells with specific specificities. In some embodiments, the specific epitope peptides comprise sequences derived from a pathogen, preferably a viral, bacterial, parasitic, or fungal pathogen, or from a cancer antigen.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-J. Genetic identification of NHC-I ligands using the EpiScan platform. (A to D) Schematic representation of the EpiScan approach. In wild-type cells (A), proteasome-derived peptides are imported into the ER by the TAP complex, trimmed by the N-terminal peptidases ERAP1 and ERAP2 and loaded onto MHC-I molecules for presentation on the cell surface. In the absence of TAP (B), however, MHC-I peptide loading is impaired; empty MHC-I molecules remain in the ER and cell surface MHC-I levels decrease. Under these conditions, delivery of exogenous peptide into the ER that binds MHC-I restores cell surface MHC-I levels (C). Exogenous peptides are targeted to the ER using the lentiviral EpiScan vector (D), which expresses a putative MHC-I ligand downstream of a signal peptide. (E to J) Validation of the EpiScan approach. EpiScan cells expressing either a humanized H2-K^{b} allele (E and F), HLA-A2 (G and H) or HLA-A3 (I and J) were transduced with the EpiScan vector expressing the indicated peptides and cell surface MHC-I levels were measured by flow cytometry. Representative histograms are shown in (E), (G) and (I); the data shown in (F), (H) and (J) represent the mean±SEM of the fold change in mean fluorescence intensity (MFI) relative to the average of the negative control for that experiment. Each dot represents a different biological replicate. Peptides shown in blue represent negative controls; peptides shown in red or orange represent positive controls. Peptides are color-coded such that histograms display representative data of the corresponding dot plot results. (****P<0.0001, *P<0.05 relative to the PRKLPKLGP (SEQ ID NO:153) negative control peptide, one-way ANOVA with Dunnett's multiple-comparison test). Sequences shown include: 1F: SIINFEKL (SEQ ID NO:33), QLESIINFEKL (SEQ ID NO:154), LEQLESIINFEKL (SEQ ID NO:155), NLVPMVATV (SEQ ID NO:34), PRKLPKLGP (SEQ ID NO:153), RDGCK (SEQ ID NO:156), and SLLNATAIAV (SEQ ID NO: 157); 1H: ANLVPMVATV (SEQ ID NO:158), QAGILARNLVPMVATV (SEQ ID NO: 159); and 1J: ALNFPGSQK (SEQ ID NO: 160) and ILRGSVAHK (SEQ ID NO: 170).

FIGS. 2A-C. EpiScan pooled screening allows high-throughput NHC-I ligand discovery. (A) Schematic representation of the screening procedure. A pool of random oligonucleotides encoding 9-mer peptides were cloned into the EpiScan lentiviral vector and expressed in EpiScan cells expressing a single HLA allele. Cells expressing exogenous peptides binding MHC-I that hence exhibited elevated cell surface MHC-I levels were isolated by FACS and the identity of the peptides revealed by next-generation sequencing. The left dot plot displays two separate samples; light grey dots are the negative control EpiScan cells without the library to demonstrate differences in GFP and surface MHC-I from the dark grey dots, which are library-containing cells. (B and C) EpiScan screens recapitulate known binding preferences for common MHC-I alleles. Logoplots summarize the sequences of the MHC-I ligands identified by EpiScan (B); for comparison, analogous logoplots based on MHC-I ligands identified by mass spectrometry⁴ are shown in (C).

FIGS. 3A-F. EpiScan and mass spectrometry represent complementary approaches for NHC-I ligand identification. (A) EpiScan- and MS-identified peptides reveal similar MHC-I binding preferences. Clustergram represents the pairwise correlation coefficients comparing the MHC-I ligands identified by EpiScan (ES) and MS; correlations were calculated by linearizing a matrix of amino acid frequencies for each of the nine positions of the peptides. (B and C) Effective detection of cysteine-containing MHC-I ligands by EpiScan. Cysteine is greatly enriched among MHC-I ligands identified by EpiScan compared to MS (B). Whilst cysteine is observed at approximately the expected frequency across MHC-I ligands identified by EpiScan, cysteine is depleted across all positions if MS-identified MHC-I ligands (C). (D) Individual EpiScan validation that cysteine-containing peptides bind HLA-A3. The indicated peptides, that were not predicted to bind HLA-A3 by NetMHC, were introduced into HLA-A3-expressing EpiScan cells and cell surface MHC-I levels measured by flow cytometry. Positive and negative control peptides are shown in red and blue respectively. (E and F) Computational prediction of MHC-I ligands using EpiScan data. Schematic representation of the neural network architecture (adapted from ⁴) (E), and comparison of the predictive power of the EpiScan models compared to the MSi models⁴(F).

FIGS. 4A-G. Comprehensive identification of NHC-I ligands expressed by SARS-CoV-2. (A to C) EpiScan analysis of the SARS-CoV-2 immunopeptidome. All possible 9-, 10- and 11-mer peptides encoded by the SARS-CoV-2 genome (A) were synthesized via an oligonucleotide array, cloned into the lentiviral EpiScan vector, and MHC-I ligands identified by the EpiScan screening procedure described previously (B). In total, 11 alleles were screened; the proportion of the US population represented by these alleles is indicated in (C). (D) Analysis of Spike (S) gene conservation among coronaviruses relative to the position of high-confidence MHC-I peptides. Along the top, symbols denote the location of peptides in the S sequence for each allele. The bottom is a aa rolling average of the S conservation score, a higher number meaning more conserved. (E and F) SARS-CoV-2 EpiScan screen results for HLA-A*02:01. (E) Scatterplot showing HLA-A2 peptide ligands concordantly identified across screen replicates. (F) Individual validation of screen hits in the EpiScan assay. The indicated peptides were introduced into HLA-A*02:01-expressing EpiScan cells and an increase in cell surface MHC-I was measured by flow cytometry. (G) Convalescent COVID-19 patients harbor CD8⁺ T cells specific for HLA-A*02 ligands identified by EpiScan. Bar plot values are the percent tetramer positive CD8⁺ T cells subtracted by the median value for each patient. Fluorescently-labeled MHC-I tetramers loaded with the indicated peptides (in colors, matched to bar blot) were used to identify reactive T cells isolated from peripheral blood. Grey dots denote control tetramer staining.

FIGS. 5A-F. Validation of successful CRISPR/Cas9-mediated disruption of HLA-I, TAP1/2 and ERAP1/2 and signal peptide testing. (a) Histogram depicting the relative amounts of surface MHC-I comparing parental HEK-293T cells, the TAP1/2 knockout clone and cells expressing the BoHV-1 UL49.5 gene, which inhibits the TAP complex (8). (b) Immunoblot validation of CRISPR-Cas9 mediated knockout of ERAP1; GAPDH was used as a loading control. (c) Sanger sequencing of the ERAP2 locus targeted by CRISPR-Cas9. The locus was amplified by PCR and the products cloned into ZeroBlunt TOPO vectors and Sanger sequenced. ERAP2 KO clone 6 exhibited a 221 bp deletion in all 11 sequenced clones. (d) Histograms depicting the relative amounts of surface MHC-I, as determined by β2M staining, between parental 293T cells and the HLA-I KO clone. (e) Testing signal peptides for the delivery of exogenous peptides to the ER. HEK-293T cells lacking TAP1/2 were infected with vectors expressing the indicated peptides fused to the following signal peptides: Env, signal peptide from the gp70 gene of mouse mammary tumor virus (8); mmIgK, modified murine Kappa Immunoglobulin signal peptide (9); and Azuro, signal peptide from the human Azurocidin preproprotein (9). Sequences highlighted in green indicate positive controls, while sequences highlighted in red indicate negative controls. (f) EpiScan with viral TAP inhibition instead of CRISPR-KO. 293 Ts were infected with lentivirus encoding a viral TAP inhibitor, UL49.5³⁸⁻⁴⁰. Then, EpiScan vectors encoding the indicated peptides were introduced via lentivirus into the UL49.5-bearing 293 Ts. Cells expressing both UL49.5 and the peptides were subjected to flow cytometry after staining with an HLA-A02-specific antibody. The bars and whiskers represent the mean±SEM of the fold change in mean fluorescence intensity (MFI) relative to the average of SIINFEKL (SEQ ID NO:33) for that experiment. Each dot represents a different biological replicate. SIINFEKL (SEQ ID NO:33) vs. SLLNATAIAV (SEQ ID NO:157) and SIINFEKL (SEQ ID NO:33) vs. VLYQDVNCTEV (SEQ ID NO:23) have p-values of <0.0001 and 0.0839, respectively, via one-way ANOVA with Dunnett's multiple-comparison test. Data are represented as mean±SEM of the fold change in mean fluorescence intensity (MFI) relative to the average of the negative controls for that experiment. Each dot represents a different biological replicate. ****p<0.0001 for each group relative to RFP by one-way ANOVA with Dunnett's multiple-comparison test.

FIGS. 6A-D. Examining the role of ERAP1 and ERAP2 in the processing of exogenous peptides delivered to the ER. EpiScan cells expressing the indicated MHC-I alleles were transduced with the indicated peptides and MHC-I levels assessed by flow cytometry using the indicated antibodies. The cells used in (a) lack ERAP1 and ERAP2; in (b) ERAP1 was re-expressed, in (c) ERAP2 was re-expressed and in (d) both ERAP1 and ERAP2 were re-expressed. Data are represented as mean±SEM of the fold change in mean fluorescence intensity (MFI) relative to the average of two negative control peptides, PRKLPKLGP (SEQ ID NO:153) and RDGCK (SEQ ID NO:156). Each dot represents a different biological replicate. *p<0.05, **p<0.01, ***p<0.001, ****p<0.0001 for each group relative to the RDGCK (SEQ ID NO:156) peptide by one-way ANOVA with Dunnett's multiple-comparison test.

FIGS. 7A-D. Peptide pulsing experiments in TAP-deficient cells. Cells were plated into serum-free media and pulsed with peptide at the indicated concentration for 24 h, and then subjected to flow cytometry to measure cell surface MHC-I levels. (a) HEK-293T TAP KO cells expressing H2-K^{b}, or a humanized version of the murine H2-K^{b} wherein the β2M interacting domain was replaced with the human equivalent; a pan-H2 antibody was used for flow cytometry. (b) The indicated HLA-A2-expressing cell lines were stained with A2 antibody. (c) The indicated HLA-A3:-expressing cell lines were stained with a pan-HLA-I antibody. (d) The indicated HLA-A3-expressing cell lines were stained with a pan-HLA-I antibody. For all panels, data are represented as mean SEM of the fold change in mean fluorescence intensity (MFI) relative to the average of the vehicle controls. *p<0.05, **p<0.01, ****p<0.0001 for each group relative to vehicle control by one-way ANOVA with Dunnett's multiple-comparison test.

FIGS. 8A-I. Sorting strategy for the random 9-mer EpiScan screens. EpiScan cells were transduced with the random 9-mer library, selected with puromycin and sorted into four bins. After five days in culture, the sorted cells were stained and analyzed by flow cytometry to assess enrichment elevated cell surface MHC-I. (a) First, cells are gated away from debris. (b) Doublets are excluded. (c) Dead cells (propidium iodide positive) are excluded. (d) Cells expressing the EpiScan vector (GFP positive) are selected. The alleles assayed were (e) HLA-A*02:01, (f) HLA-B*08:01, (g) HLA-A*03:01, (h) HLA-B*57:01, and (i) HLA-B*57:01 after 48 h abacavir treatment at 6 μM. All allele screens were done twice, except HLA-B*57:01, which was done only once.

FIGS. 9A-D. Summary of the properties of the NHC-I ligands identified by the SARS-CoV-2 EpiScan screens across 11 MHC-I alleles. (a) Length distribution of peptide binders. (b) ORF length (left y-axis) versus high-confidence binders per ORF (right y-axis). (c) The number of high-confidence binders per allele; cysteine-containing peptides are highlighted in purple. (d) Positive predictive value of ESP models when applied to SARS-CoV-2 EpiScan screening data.

FIG. 10. Comparisons of EpiScan signal:noise for various HLA-I alleles with and without HM13 knockout. The data shown represent the mean±SEM of the fold change in mean fluorescence intensity (MFI) relative to the average of the negative control for that experiment. Each dot represents a different biological replicate. The leftmost two, four, three and three peptides represent positive controls for A*02, A*03, B*08, B*57, respectively. All other peptides are negative controls.

FIG. 11. Comparison of affinity of L- to V-ended 9mers via EpiScan. The data shown represent the mean±SEM of the fold change in mean fluorescence intensity (MFI) of the V-ended relative to the L-ended versions of the peptide sequences indicated below. Each dot represents a different biological replicate.

FIG. 12. Confirmation of signal peptidase cleavage fidelity. The data shown represent the mean±SEM of the fold change in mean fluorescence intensity (MFI) relative to the average of the negative control for that experiment. Each dot represents a different biological replicate. The peptides listed below represent the wildtype (WT) sequence, squares, which was compared to a one amino acid N-terminal truncation (circles), and addition of an N-terminal glycine (triangles).

FIG. 13. SARS-CoV-2 HM13 KO EpiScan screen results for HLA-A*02:01. Scatterplot showing HLA-A2 peptide ligands concordantly identified across screen replicates. Bolded sequences represent those we have identified reactive T-cells in convalescent COVID-19 patients. Underlined sequences represent other publications have identified reactive T-cells in convalescent COVID-19 patients.

FIG. 14. Individual validation of screen hits in the EpiScan assay. The indicated peptides were introduced into the indicated EpiScan cells and an increase in cell surface MHC-I was measured by flow cytometry. The data shown represent the mean±SEM of the fold change in mean fluorescence intensity (MFI) relative to the average of the negative control for that experiment. Each dot represents a different biological replicate. All comparisons are statistically significant at p<0.0001 by one-way ANOVA with Dunnett's multiple comparisons correction unless indicated (***=p<0.001, **=p<0.01 and n.s.=not significant).

FIG. 15. EpiScan screens can be performed by magnetic-activated cell sorting (MACS). A diverse set of 200,000 distinct peptides was introduced into HLA-A*02:01 HM13 KO EpiScan cells. After selection, MACS was performed using a biotin-conjugated B2m antibody on 100 million cells for each condition, and the column flow through and the cells captured by the column were plated after sorting. Two days later the cells were stained with APC-anti-HLA-A*02:01 antibody and an increase in cell surface MHC-I was measured by flow cytometry.

FIG. 16. EpiScan can be used to directly elicit CD8 T-cell responses. For this experiment, primary T cells were infected with a TCR, NLV3, that is specific to the peptide NLVPMVATV, then those T cells were incubated together for 16 h at a 1:1 ratio with the EpiTScan cells that express NLVPMVATV (Epi pp65, far left) via the EpiScan Vector, or two negative control peptides via the EpiScan Vector (Epi SAV10 and SIIN), no peptide at all (neg), or NLVPMVATV was added directly to the media (pulsed pp65). In the top graph, the Granzyme reporter in the EpiTScan cells is being measured. As expected, both pulsed peptide and EpiScan Vector expressed pp65 cause the NLV3 T cells to activate the GzB reporter. The bottom two graphs are different measures of T cell activation. The middle, trogocytosis, is measured by the transfer of BFP from the cytoplasm of EpiTScan cells to the T-cells; BFP transfer indicates successful synapse formation between the T cell and the EpiTScan cells. CD69 (bottom) is a T cell activation marker. Here, CD69 surface staining on the T cells was highest in the pp65 conditions.

### DETAILED DESCRIPTION

Described herein are cell-based genetic methods, one example of which is referred to herein as ‘EpiScan,’ that allow for rapid empirical determination of MHC-I binding for large pools of peptides, leveraging inexpensive DNA oligonucleotide synthesis to generate pre-defined libraries for targeted immunopeptidomics. The system can be used for querying individual peptides for MHC-I binding.

The present methods rely on the fact that HLA-I proteins are only stable on the cell surface when bound to a peptide. Thus, if a cell expressing only one HLA-I gene and one candidate peptide has HLA-I on its surface, as identified by flow cytometry, then that HLA-I protein must have bound to that peptide. However, a typical mammalian cell expresses several HLA-I genes/alleles and each HLA-I allele is exposed to tens of thousands of potential peptides. Thus, provided herein are cells engineered to remove expression of one, two, three, four, or more, e.g., all, relevant immune presentation related genes (e.g., HLA-A, B and -C; TAP1 and -2; ERAP1 and -2, and signal peptide peptidase HM13). In some embodiments, one or more or all of HLA-E, -F and -G are also deleted. TAP1/2 deletion prevents cytosolic peptides from being transported into the ER. ERAP1/2 deletion prevents ER-resident peptides, such as signal peptides, from being further processed to a length more suitable for HLA-I binding. HM13 deletion prevents membrane-resident signal peptides from being cleaved and released into the ER. The cells are also engineered to express only one HLA-I gene/allele, e.g., to retain a single endogenous HLA-I allele (e.g., one of HLA-A, -B, -C, -E, -F, or -G), or a single HLA-I allele can be introduced, e.g., via viral, preferably lentiviral, transduction. Cells lacking one or more of these genes would facilitate the detection of HLA driven to the surface by peptides engineered to go directly to the ER for loading onto HLA. A number of methods are known in the art for knocking out genes, including the use of CRISPR-Cas or other RNA-guided nucleases, TALEs, or zinc fingers, to introduce mutations that abrogate expression of a target gene, e.g., by introduction of a mutation that inserts a stop codon resulting in expression of a non-functional fragment of the target gene, or by homologous recombination to delete all or a part of the target gene. Alternatively, other methods can be used to reduce or eliminate expression of the genes. For example, for TAP knockout, viral TAP inhibition can be used instead of CRISPR-KO; for example a viral TAP inhibitor, UL49.5³⁸⁻⁴⁰, can be used. In addition, viral gene induced degradation of HLA can be used. These methods include introduction of a viral gene such as human cytomegalovirus (HCMV) US2 or US11 (see Van den Boomen and Lehner, Mol. Immunol. 68, 106-111 (2015)), which use mammalian ER-associated degradation (ERAD) to induce rapid degradation of major histocompatibility class I (MHC-I) molecules, thereby degrading endogenous HLA alleles. Next, an HLA allele of choice that no longer has the lysine residue/s upon which US2 or US11 cause ubiquitination then degradation. Thus, the introduced allele is the only one that is not degraded. In some embodiments, expression of TAP genes is reduced using viral TAP inhibitor UL49.5, and expression of HLA is reduced using HCMV US2 or US11, thereby obviating the need for genomic engineering methods such as CRISPR to create a cell line.

In addition, a number of methods are known in the art for introducing a sequence into a cell, e.g., by use of a vector containing nucleic acid, e.g., a cDNA. The vectors can be viral vectors, including recombinant retroviruses (e.g., lentivirus), adenovirus, adeno-associated virus, lentivirus, and herpes simplex virus-1, or recombinant bacterial or eukaryotic plasmids. In some embodiments, transposons like Sleeping Beauty or piggyback are used, or plasmids that integrate site specifically by Cre or FLP-mediates integration or by homologous recombination into a particular locus. All of these could allow a screen to be performed at high complexity. By the way you should mention retroviruses as a class (lentivirus is a special kind of retrovirus) Viral vectors transfect cells directly; plasmid DNA can be delivered naked or with the help of, for example, cationic liposomes (lipofectamine) or derivatized (e.g., antibody conjugated), polylysine conjugates, gramacidin, artificial viral envelopes or other such intracellular carriers, as well as direct injection of the gene construct or CaPO₄ precipitation carried out in vivo. See, e.g., Hall et al., Curr Protoc Cell Biol. 2009 September; CHAPTER: Unit19.1217; Doyle et al., Transgenic Res. 2012 April; 21(2): 327-349; Jin et al., PLoS One. 2020; 15(2): e0228910. The methods can include performing sequencing assays to confirm the presence of the intended mutation; RNA assays to confirm a lack of functional transcript; or protein detection methods to confirm a lack of protein.

Exemplary human genomic sequences encoding the target proteins that can be knocked out are provided in the following table.

| Protein | Genomic sequence* |
|---|---|
| HLA-A (major histocompatibility | NG_029217.2, Range 5005-8420 |
| complex, class I, A) | |
| HLA-B (major histocompatibility | NG_023187.1, Range 5034-8338 |
| complex, class I, B) | |
| HLA-C (major histocompatibility | NG_029422.2, Range 4996-8383 |
| complex, class I, C) | |
| HLA-E (major histocompatibility | NC_000006.12, Range |
| complex, class I, E) | 30489508-30494194 |
| | (Reference GRCh38. p13 |
| | Primary Assembly) |
| HLA-F (major histocompatibility | NG_012009.1, Range 5095-8957 |
| complex, class I, F) | |
| HLA-G (major histocompatibility | NG_029039.1, Range 5001-9144 |
| complex, class I, G) | |
| TAP1 (transporter 1, ATP binding | NG_011759.1, Range 5001-13763 |
| cassette subfamily B member) | |
| TAP2 (transporter 2, ATP binding | NG_009793.3, Range 5001-2193 |
| cassette subfamily B member) | |
| ERAP1 (endoplasmic reticulum | NG_027839.2, Range |
| aminopeptidase 1) | 132795-180174 |
| ERAP2 (endoplasmic reticulum | NG_027839.2, Range |
| aminopeptidase 2) | 132795-180174 |
| HM13 (histocompatibility minor | NG_051619.2, Range 5001-60158 |
| 13) | |

*NCBI RefSeqGene unless otherwise noted

These cells thus engineered lack short peptides in the ER, and presentation on MHC-I is impaired or lost, in the absence of expression of an exogenous sequence linked to a signal peptide that directs a peptide or other sequence to the ER, as described below.

Exemplary sequences for human HLA-I proteins and cDNAs encoding the proteins are provided in the following table.

| | cDNA sequence | |
|---|---|---|
| Protein | (NCBI RefSeq) | Protein sequence |
| HLA-A | NM_001242758.1 | NP_001229687.1* |
| | NM_002116.8 | NP_002107.3** |
| HLA-B | NM_005514.8 | NP_005505.2 |
| HLA-C | NM_001243042.1 | NP_001229971.1*** |
| | NM_002117.6 | NP_002108.4 |
| HLA-E | NM_005516.6 | NP_005507.3 |
| HLA-F | NM_001098478.2 | NP_001091948.1 |
| | NM_001098479.2 | NP_001091949.1 |
| | NM_018950.3 | NP_061823.2 |
| HLA-G | NM_001363567.2 | NP_001350496.1 |
| | NM_001384280.1 | NP_001371209.1 |
| | NM_001384290.1 | NP_001371219.1 |
| | NM_002127.6 | NP_002118.1 |

*HLA class I histocompatibility antigen, A alpha chain A*01:01:01:01 precursor
**HLA class I histocompatibility antigen, A alpha chain A*03:01:01:01 precursor
***HLA class I histocompatibility antigen, C alpha chain precursor, C*07:01:01:01 allele
**** HLA class I histocompatibility antigen, C alpha chain precursor, C*07:02:01 allele

Although the sequences provided above are human, other species can also be used; so long as a beta-2-microglobulin domain that binds the MHC of interest is also introduced, then any species' MHC can be studied. For example, a humanized version of the murine H2-Kb can be used, wherein the beta-2-microglobulin (β2M) interacting domain was replaced with the human equivalent the sequence is as follows (dotted underline and bold represents “humanized sequence” that was taken from HLA-A*02:01 and the rest is from mouse H2-Kb):

(SEQ ID NO: 171)

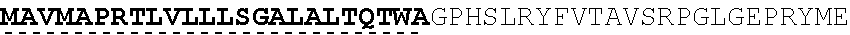

VGYVDDTEFVRFDSDAENPRYEPRARWMEQEGPEYWERETQKAKGNEQ
SFRVDLRTLLGYYNQSKGGSHTIQVISGCEVGSDGRLLRGYQQYAYDG
CDYIALNEDLKTWTAADMAALITKHKWEQAGEAERLRAYLEGTCVEWL

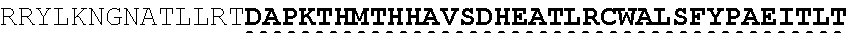

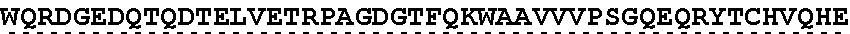

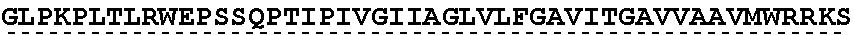

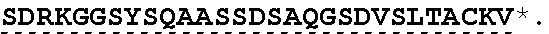

Further, although human cells are exemplified herein, other mammalian species' cells can also be used, e.g., non-human primates, cats, dogs, horses, cows, goats, sheep, stoats, and so on.

In some embodiments, the cells are also engineered to express selected candidate epitope peptides, e.g., one or more selected candidate epitope peptides, in the ER where HLA-I samples potential peptides for binding. By fusing the peptide of interest to a signal peptide, as the peptide is translated into the ER it is cleaved without needing any further processing. Preferred signal peptides include codon-optimized MMTV gp70 signal peptide (MPNHQSGSPTGSSDLLLDGKKQRAHLALRRKRRREMRKINRKVRRMNLAPIKE KTAWQHLQALIFEAEEVLKTSQTPQTSLTLFLALLAVLAPPPVSG (SEQ ID NO:172). Additionally, in preferred embodiments the signal peptide used is longer than 16 nucleotides, thus preventing its binding to HLA-I. The sequence encoding the peptide-signal peptide can be introduced into the cell, e.g., via viral, preferably lentiviral, transduction. In some embodiments, the peptide is ultimately exported from the ER; see. e.g., Byun, et al., J. Virol. 86, 214-25 (2012). Alternatively, synthesized peptides can be used with the EpiScan cells to determine MHC-I binding. The peptides can include the signal peptides. Synthetic peptides, e.g., produced using solid phase peptide synthesis (SPPS), can be added to the media; see, e.g., the “T2 assay,” Stuber et al., Eur J Immunol. 1992; 22(10):2697-2703.

MHC class I molecules are expressed in all nucleated cells and in platelets. The parental or host cells used for these methods can include any mammalian cells, preferably human cells, that can be maintained in culture. Examples of cells that can be used for the present methods and compositions include cells from cell lines, e.g., HEK-293T cells. In some embodiments, the cells are of tumor origin, or are not of tumor origin. Examples of commercially available human cell lines from non-tumor sources include CCD-1064Sk (ATCC® CRL-2076); HCC1599 BL (ATCC® CRL-2332); BJ (ATCC® CRL-2522); HCC1395 BL (ATCC® CRL-2325); HCC2157 BL (ATCC® CRL-2341) (+); COLO 829BL (ATCC® CRL-1980); HGF-1 (ATCC® CRL-2014); HCC1143 BL (ATCC® CRL-2362); Hs27 (ATCC® CRL-1634); FHC (ATCC® CRL-1831); HCC1007 BL (ATCC® CRL-2319); MRC-5 (ATCC® CCL-171); HUV-EC-C [HUVEC] (ATCC® CRL-1730); CCD-8Lu (ATCC® CCL-201); HEL 299 (ATCC® CCL-137); MCF-12F (ATCC® CRL-10783); CCD-33Lu (ATCC® CRL-1490); CCD-112CoN (ATCC® CRL-1541); Malme-3 (ATCC® HTB-102) (+); RWPE-2 (ATCC® CRL-11610); NCI-BL2126 [BL2126] (ATCC® CCL-256.1); HCC1937 BL (ATCC® CRL-2337); CCD-19Lu (ATCC® CCL-210); THLE-3 (ATCC® CRL-11233); 184B5 (ATCC® CRL-8799); CCD-986Sk (ATCC® CRL-1947) (+); HFL1 (ATCC® CCL-153); IMR-90 (ATCC® CCL-186); WPMY-1 (ATCC® CRL-2854); CCD-18Co (ATCC® CRL-1459) (+); RWPE-1 (ATCC® CRL-11609) (+); OAT1 HEK 293T/17 (ATCC® CRL-11268G-1); Detroit 548 (ATCC® CCL-116); MRC-9 (ATCC® CCL-212); NCI-BL1184 [BL1184](ATCC® CRL-5949); CCD 841 CoN (ATCC® CRL-1790); HS-5 (ATCC® CRL-11882); LL 24 (ATCC® CCL-151); HCC38 BL (ATCC® CRL-2346); NCI-BL1437 [BL1437] (ATCC® CRL-5958); Hs 895.Sk (ATCC® CRL-7636); WI-38 (ATCC® CCL-75); ARPE-19 (ATCC® CRL-2302); Detroit 551 (ATCC® CCL-110); Hs 578Bst (ATCC® HTB-125); FHs 74 Int (ATCC® CCL-241); NCI-BL1770 [BL1770] (ATCC® CRL-5960); WS1 (ATCC® CRL-1502) (+); CCD-1070Sk (ATCC® CRL-2091); CCD-16Lu (ATCC® CCL-204); NCI-BL2009 [BL2009] (ATCC® CRL-5961); HCC1954 BL (ATCC® CRL-2339); CCD-1079Sk (ATCC® CRL-2097); CCD-33Co (ATCC® CRL-1539); HCC2218 BL (ATCC® CRL-2363); NCI-BL1395 [BL1395] (ATCC® CRL-5957); Het-1A (ATCC® CRL-2692); TE 353.Sk (ATCC® CRL-7761); WPE1-NB26 (ATCC® CRL-2852); NCI-BL2052 [BL2052] (ATCC® CRL-5963); CCD-1059Sk (ATCC® CRL-2072); NCI-BL209 [BL209] (ATCC® CRL-5948); Hs 605.Sk (ATCC® CRL-7364); CCD-1090Sk (ATCC® CRL-2106); WPE1-NA22 (ATCC® CRL-2849); Hs 925.Sk (ATCC® CRL-7676); HBE4-E6/E7 [NBE4-E6/E7] (ATCC® CRL-2078); NCI-BL2195 [BL2195] (ATCC® CRL-5956); NCI-BL2087 [BL2087] (ATCC® CRL-5965); NCI-BL128 [BL128] (ATCC® CRL-5947); Hs 742.Sk (ATCC® CRL-7481); NCI-BL1672 [BL1672] (ATCC® CRL-5959); CCD-27Sk (ATCC® CRL-1475); Hs 789.Sk (ATCC® CRL-7518); WPE1-NB14 (ATCC® CRL-2850); and WPE1-NB11 (ATCC® CRL-2851). Other cell lines that can be used include lymphoid derived cells, e.g., K-562 (ATCC® CCL-243) or SKW 6.4 (ATCC® TIB-215). In some embodiments, the cell is a B cell or B-lymphoid cell, or is derived from an immortalized B cell (see, e.g., Nilsson et al., Hum Cell. 1992 March;5(1):25-41). In some embodiments, the cell is a K-562 cell that expresses GM-CSF (e.g., Smith et al., Clin Cancer Res. 2010 Jan. 1; 16(1): 338-347). In some embodiments, the cells are T2 or RMA-S (mouse), which have no TAP1/2, or B721.221, which is MHC-I deficient.

Assays

The cells described herein can be used to identify MHC-I binding epitopes. Generally speaking, in these assays a pool of oligonucleotides encoding potential MHC-I binding peptides, e.g., 8-12mer peptides, e.g., 9-mer peptides, is expressed in the cells, such that each cell expresses only one peptide (fused to a signal peptide as described above) designed to directly load onto MHC after minimal processing upon ER entry. In some embodiments, wherein ER proteases such as ERAP1/2 have been ablated, the peptide is only processed by the signal peptide peptidase to release it from the signal peptide. Alternatively, endogenous proteases can still be active and the peptide is further processed in the ER prior to binding to MHC. Additionally, exogeneous proteases may be introduced that can process the peptide. One could also modify genes in the peptide loading complex, such as TAPBP, CALR or PDIA3. In some embodiments, the oligonucleotides are random. In some embodiments, at least 1; 5; 10; 100; 1,000; 10,000; 100,000; 200,000; 250,000; 300,000; or more different peptides are sampled. The cells can be assayed as a pool in a unified sample, wherein the sample includes a plurality of different clones, each clone expressing different peptides. In some embodiments, the methods are used to identify MHC-I binding epitopes, and the pool of oligonucleotides comprises every possible 8-12mer peptide in a selected protein representing every possible 8-12mer from the selected protein. Alternatively, the oligonucleotides can represent a curated selection of 8-12mers, e.g., from candidate portions of the selected protein that are better candidates for MHC-I binding. Such candidate portions can be identified using methods known in the art, e.g., bioinformatics methods such as etMHC 4.0, NetMVHC 3.4, NetMHCpan 4.0, NetMHCpan 3.0, NetMHCpan 2.8, NetMHCcons 1.1, PuickPocket 1.1, IEDB recommended, IEDB consensus, IEDB SMMPMBEC, IEDB SMM, MHCflurry 1.1, and SYFPEITHI; see Bonsack et al., Cancer Immunol Res May 1 2019 (7) (5) 719-736.

Sequences encoding the peptides are cloned into an expression vector comprising a promoter for expression of the peptides, e.g., the exemplary EpiScan lentiviral vector described herein, and expressed in cells expressing a single HLA allele with modifications to HLA-I presentation machinery described herein. Cells expressing exogenous peptides that bind MIHC-I exhibit elevated cell surface MIHC-I levels, and can be isolated, e.g., using fluorescence-activated cell sorting (FACS) or magnetic-activated cell sorting (MACS). Then, the identity of the peptides can be determined, e.g., by sequencing, e.g., next-generation sequencing, using primers that bind to the vector sequence on either side of the sequence encoding the peptide.

In some embodiments, variants of (i.e., at least 60, 70, 80, 85, 90, 95, 97, 99% identical to) the proteins and nucleic acids described herein can be used. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Applications

The present methods and compositions have many applications in both basic and translational research, as well as clinical practice.

As demonstrated with SARS-CoV-2, the present methods can be used for uncovering the entire MHC-I immunopeptidome for a single protein or pathogen, e.g., to identify MHC-I binding epitopes in one or more proteins from a pathogen, e.g., a bacterium, virus, parasite, or fungus. Once the epitopes have been identified, cells can be engineered to express one or more of the epitopes for use in a live cell vaccine, and administered to a subject to elicit an immune response to the pathogen from which the epitope was derived.

The present methods can also be used to generate cells that display only, or a majority of (e.g., at least 1%, 2% 5%, 10%, 20%, 30%, 40%, 50%, 60%, 70%, 80%, 90%, or 95%) a single peptide. In this way, dendritic cells that display only, or a large majority of, a single peptide, which can be used to focus a vaccine on a subset of epitopes. Cells that express a single, or a majority of, single peptides can be used to isolate rare T-cells specific to that peptide:MHC complex.

These methods can be used to find potential epitopes in any given protein. Once identified, the present methods can include using known molecular biology methods to ‘deimmunize’ the protein,³⁰ e.g., by mutation of identified epitopes until the epitope is no longer presented. The mutated proteins (or mutated peptides therefrom) can then be subjected to further rounds of epitope scanning to confirm reduction or loss of MHC binding epitope. These methods can be used to develop nonimmunogenic gene therapies, e.g., for humans.

Classical vaccination methods utilize immunization with full-length proteins, but the immune response that follows typically focuses on only a subset of potential antigenic epitopes through the poorly understood process of T cell immunodominance³¹. Knowledge of the assortment of potential T cell epitopes given the MIHC-I haplotype of any given individual could guide the development of personalized vaccines, which should provide a broader and potentially more durable response³². In particular, the present methods can be used for assessment of potential neo-antigen peptide:MHC-I complexes necessary for personalized cancer vaccines³³. The methods can be used to test recurrent cancer mutations for HLA display to match neoantigens and HLAs. In addition, the methods can be used to profile patient specific cancer mutations for HLA display.

The methods can also be used to identify tissue- or pathology-specific peptides presented on MHC to later use as vaccine targets.

In addition, the methods can be used to screen for interventions such as viruses, proteins, genes, or small molecules that enhance binding of a particular peptide on a given HLA, block HLA binding or that change the specificity of an HLA. These methods include conducting the assays described herein in the presence and absence of the intervention.

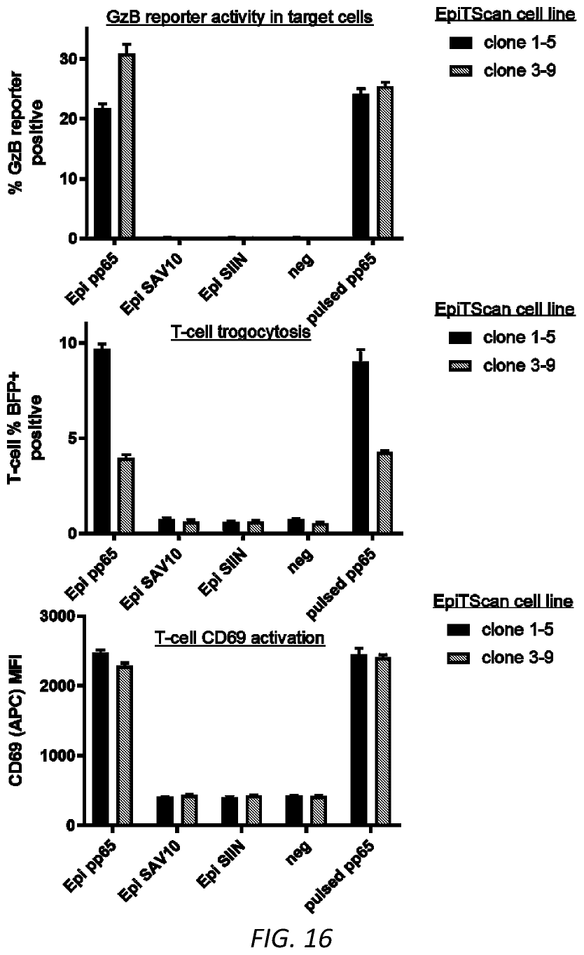

The methods can also be used to elicit T cell responses in order to precisely identify the epitope of a specific T-cell receptor (TCR). Co-incubation of EpiScan cells that express a single peptide:MHC-I complex on the surface, or pools of EpiScan cells that express different single peptide:MHC-I complexes on the surface, with T cells will activate T cells with TCRs that recognize the presented peptide:MHC-I complex. Methods known in the art, such as, but not limited to, IL-2 ELISpot (Ranieri et al., Methods Mol Biol. 2014; 1186:75-86), T-Scan (Kula et al., Cell. 2019 Aug. 8; 178(4):1016-1028.e13), CD69 FACS (Simms and Ellis, Clin Diagn Lab Immunol. 1996 May; 3(3):301-4) can be used to detect and isolate activated T cells, and the epitope can then be identified as above. See, e.g., Example 10 and FIG. 16.

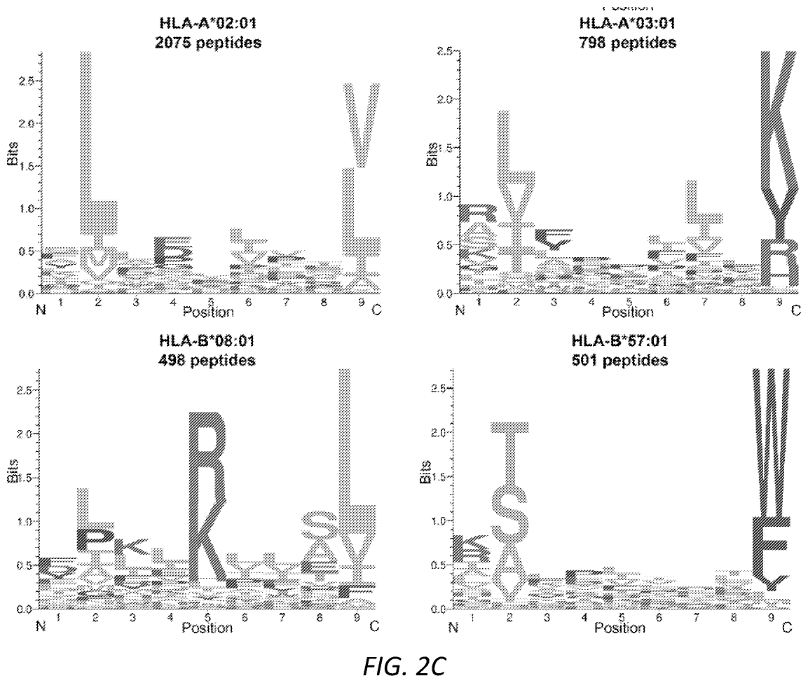

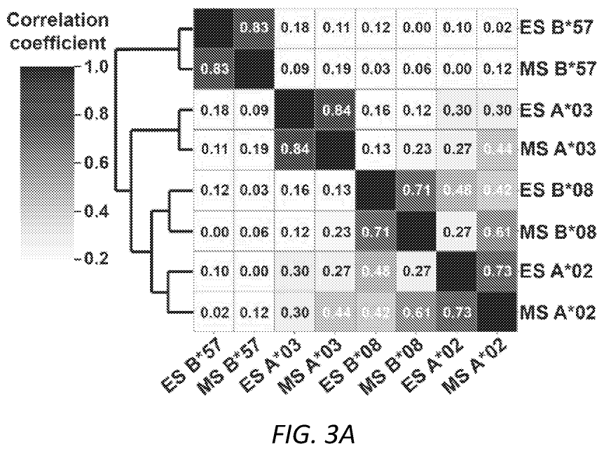

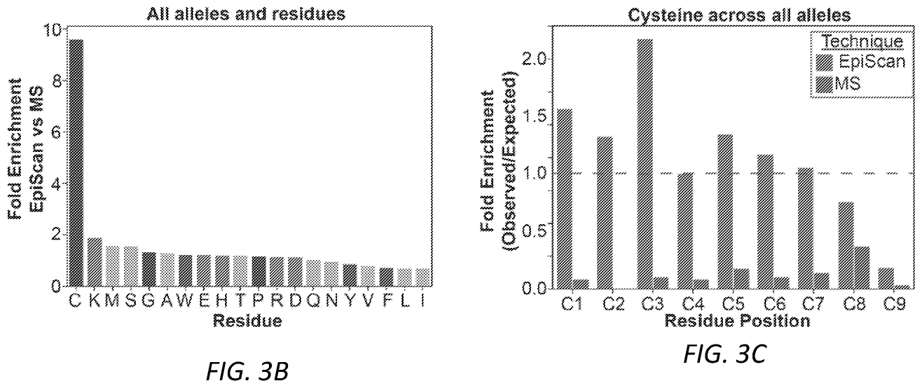

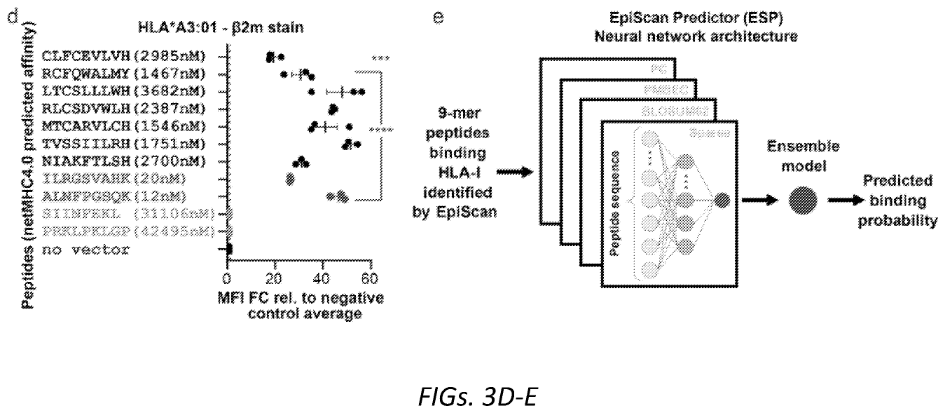

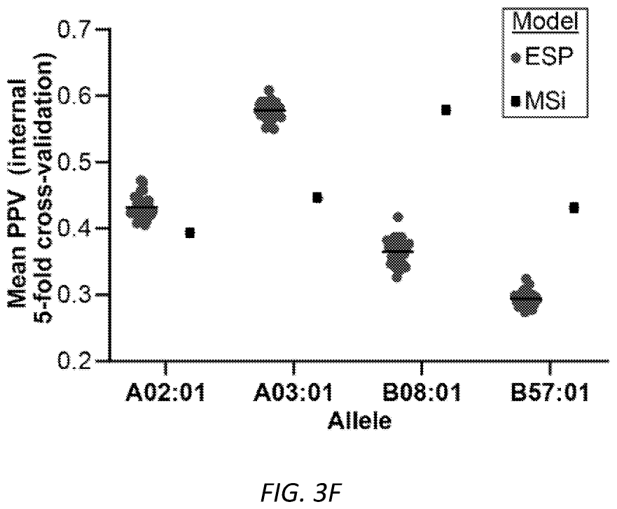

In addition, EpiScan data can be used to generate predictions about MIHC-I peptide binding preferences, for the development of computational models that can accurately predict MIHC-I ligands starting from the primary sequence of a protein^{4,20,21}. An effective prediction algorithm analogous to the MSi algorithm recently developed by Sarkizova and colleagues⁴ was developed. Machine learning models were trained to classify 9-mer peptide sequences as binders or non-binders for HLA-A2, HLA-A3, HLA-B8 and HLA-B57. In addition to not suffering from detection bias inherent to MS, these methods render predictions solely based on allele-specific affinity, and thus can identify MHC-I ligands that aren't subject to proteasome processing or TAP import. See, e.g., Example 3 and FIGS. 3E-F.

### EXAMPLES

The invention is further described in the following examples, which do not limit the scope of the invention described in the claims.

Materials and Methods

The following materials and methods were used in the Examples below.

Cell Culture

HEK-293T (CRL-3216), T2 (CRL-1992) and CIR (CRL-2369) cells were obtained from ATCC. T2 and C1R cells were cultured in IMDM (Gibco, 12440053) with 10% FBS (HyClone) and 1% penicillin-streptomycin (15140-122, Invitrogen); HEK-293T were cultured in 10% DMEM (Gibco, 11995065) with 10% FBS (HyClone) and 1% penicillin-streptomycin (15140-122, Invitrogen). All cell lines were regularly tested for mycoplasma and all negative.

Generation of EpiScan Cells

HEK-293T cells were transfected with sgRNAs targeting TAP1 and TAP2; cells exhibiting diminished cell surface MHC-I were then single cell cloned by sorting into 96-well plates. An MHC-I^{low} clone was then transfected with two sgRNAs targeting all endogenous MHC-I alleles. Cells lacking any detectable cell surface MHC-I were then single cell cloned. Then, a TAP1/2 deficient, MHC-I null clone was transfected with sgRNAs targeting ERAP1 and ERAP2 and single cell clones again generated from the resulting population. Successful disruption of ERAP1 and ERAP2 was confirmed by immunoblot and TOPO cloning and Sanger sequencing, respectively. Finally, cells without MHC-I, TAP1/2 or ERAP1/2 were transfected with sgRNA targeting HM13. Knockout of HM13 was confirmed via TOPO cloning and Sanger sequencing.

All sgRNAs were cloned into either lentiCRISPR v2-FE or PX458 (Addgene #48138); sequences used were:

| sgRNA name | sgRNA target sequence | SEQ ID NO: |
|---|---|---|
| sgTAP1-1 | GCCATGCGAGAGAAGCTCCG | 1 |
| sgTAP1-2 | AGTTCGAAGCTTTGCCAACG | 2 |
| sgTAP2-1 | ATCCCCATATATGTATACCA | 3 |
| sgTAP2-2 | ACAACAAAGTCTTGATGTGG | 4 |
| sgPan-MHC-I 1 | CGGCTACTACAACCAGAGCG | 5 |
| sgPan-MHC-I 2 | GAGATCACACTGACCTGGCAG | 6 |
| sgERAP1-1 | AGATTATGCACTGGATGCTG | 7 |
| sgERAP1-2 | GTGCAATTTGCTCCTGACGG | 8 |
| sgERAP1-3 | AAGGCCATTCTAGCTGCAGT | 9 |
| sgERAP2-1 | GAGATGCAACAAAGTCCAGAG | 10 |
| sgERAP2-2 | GCCTCACCTGAAATACTATG | 11 |
| sgHM13-1 | GCCCCACCAACAGCACTACG | 12 |
| sgHM13-2 | AGAAATACATGGACAGCAGG | 13 |
| sgHM13-3 | GGTATTTGGCACCAATGTGA | 14 |

Alternatively, for TAP knockout, viral TAP inhibition was used instead of CRISPR-KO. 293 Ts were infected with lentivirus encoding a viral TAP inhibitor, UL49.5³⁸⁻⁴⁰.

Generation of EpiScan Vector

A lentiviral pHAGE vector with a CMV promoter plus an EF1α promoter driving EGFP-P2A-Puro^{R} was used as the backbone. The vector was digested with PstI and AgeI to excise the EF1α promoter, and the Gibson assembly method used to insert a gBlock (IDT) encoding (1) a codon-optimized MMTV gp70 signal peptide (MPNHQSGSPTGSSDLLLDGKKQRAHLALRRKRRREMRKINRKVRRMNLAPIKE KTAWQHLQALIFEAEEVLKTSQTPQTSLTLFLALLAVLAPPPVSG (SEQ ID NO:15)), (2) filler region flanked by BsmBI sites and (3) an IRES element. The resulting vector was then converted into a Gateway-like destination vector by inserting the Cm^{R} and ccdB cassettes into the SphI site located in the filler region.

Peptide Pulsing

Cells were washed with PBS three times to remove FBS, resuspended in IMDM with 1% penicillin-streptomycin (15140-122, Invitrogen) without FBS, and 100,000 cells seeded per well of a 96-well plate. Peptides were added 24 h before analysis by flow cytometry.

Flow Cytometry

Cells were stained for at least 30 m in PBS, washed in PBS and then analyzed with a BD LSR2. All antibodies were from BioLegend and used at 1:100:
141605—APC anti-mouse H-2Kb bound to SIINFEKL (SEQ ID NO:33) Antibody,
3433051—PE anti human HLA-A2 antibody,
316317—PE/Cy7 anti-human 02-microglobulin Antibody,
141603—PE anti-mouse H-2Kb bound to SIINFEKL (SEQ ID NO:33) Antibody,
311410—APC anti-human HLA-A,B,C Antibody,
316312—APC anti-human 02-microglobulin Antibody,
125506—PE anti-mouse H-2 Antibody,
343308—APC anti human HLA-A2 antibody.
   Analysis was performed using FlowJo v10.6.1 (BD).

FACS

For EpiScan screens, 30 μl of antibody (APC-conjugated anti-human HLA-A2 antibody, BioLegend, 343308 or APC-conjugated anti-human 02-microglobulin antibody, BioLegend, 316312) in a total volume of 1.5 ml was used per 10 million cells. Staining was conducted for 30 min at 4° C.; cells were then washed in PBS prior to sorting. Sorting was performed on a Sony MA900 instrument.

Immunoblotting

Cells were pelleted, washed in PBS, and then lysed in RIPA buffer. Lysates were mixed with Novex Tris-Glycine SDS Sample Buffer containing β-mercaptoethanol and resolved on a 4-20% Tris-Glycine SDS-PAGE gel. Antibodies used were anti-GAPDH (sc-47724, Santa Cruz, 1:200) and anti-ERAP1 (MABF851, Millipore, 1:1000).

Transfection and Single Cell Cloning

HEK-293T cells were transfected using PolyJet (SignaGen, SL100688) as recommended by the manufacturer. Single cell cloning was carried out after 7 d by FACS using a Sony MA900 instrument.

Lentiviral Transduction

293T cells were transfected with PolyJet (SignaGen, SL100688) according to manufacturer's directions using a 1:1 ratio of lentiviral plasmids to packaging vectors (encoding VSV-G, Tat, Rev and Gag-Pol). Viral supernatants were harvested at 48 h and 72 h post-transfection, passaged through a 0.45 μm filter, and applied to target cells for 48 h in the presence of 8 μg/ml polybrene. Transduced cells were selected with 2 μg/ml puromycin for at least four days.

EpiScan Library Generation

Random 9-mer library. An oligo of the follow sequence was ordered from Integrated DNA Technologies: ccacctgtgagcgggNNBNNBNNBNNBNNBNNBNNBNNBNNBtaaGCacgttactgg (SEQ ID NO: 16), wherein B is Guanine/Thymine/Cytosine. It was amplified by PCR using the primers, tggccgtattggccccgccacctgtgagcggg (SEQ ID NO: 17) and attccaagcggcttcggccagtaacgtGCtta (SEQ ID NO: 18), and then cloned into the EpiScan vector digested with BsmBI using the Gibson assembly method. The resulting plasmids were then electroporated into Electromax DH10B competent cells (ThermoFisher Scientific).

SARS-CoV-2 library. Protein sequences of SARS-CoV-2 available as of 2/06/20 were downloaded from the NCBI Severe acute respiratory syndrome coronavirus 2 data hub. This represented a total of 11 strains of SARS-CoV-2. All protein sequences were broken into 9-, 10- and 11-mer fragments and duplicates were removed. The remaining sequences were then reverse translated using a custom script written in MATLAB R2019b to avoid restriction sites for EcoRI/XhoI/BsmBI/BbsI and to ensure GC content between 30% and 70%. Sequences were amplified from a SurePrint Oligonucleotide Library (Agilent) and digested with BbsI to liberate sticky ended peptide-encoding fragments. The EpiScan vector was digested with BsmBI to generate compatible sticky ends and the fragments were cloned in via T4 ligation. The ligation products were then electroporated into Electromax DH10B competent cells (ThermoFisher Scientific).

NGS Library Preparation

Genomic DNA was isolated via phenol/chloroform extraction. EpiScan vector sequences were amplified (F: tccctacacgacgctcttccgatctTACAGCTcgccacctgtgagcggg (SEQ ID NO: 19) and R: ggcttcggccagtaacgtgc (SEQ ID NO:20); the bold uppercase sequence represents a 0-7 nt variable stagger region) in a 125 μl reaction with 5 μg gDNA. PCR reactions for each sample were pooled, purified using the Machery-Nagel PCR clean-up kit (Takara, 740609), and 400 ng used for a second round of PCR to add Illumina P5 and P7 sequences and indices for multiplexing (F: aatgatacggcgaccaccgagatctacactcttTCCCTACACGACGCTCT TCCG (SEQ ID NO:21) and R: caagcagaagacggcatacgagat[xxxxxxx]GTGACTGGA GTTCAGACGTGT (SEQ ID NO:22); where [xxxxxx] represents the sample index). Finally samples were pooled, gel purified and then sequenced using an Illumina NextSeq or NovaSeq instrument.

Expression Vectors

All cDNAs were cloned into expression vectors via Gateway Cloning (ThermoFisher). ERAP1 (IOH80668) was obtained from the Harvard ORFeome v8 collection. ERAP2 and MHC-I alleles were codon optimized and synthesized as gBlocks with flanking attB sites by Integrated DNA Technologies. Destination vectors all used the EFlu promoter to drive cDNA expression and contained a selectable marker (BFP, mAmetrine, tdTomato or Hygro^{R}) driven by the PGK promoter.

Computational Prediction of NHC-I Ligands

The Keras Python library was used to train machine learning models to predict the likelihood of any given 9-mer binding MHC-I. A neural network architecture analogous to that developed by Sarkizova and colleagues⁴ was employed, with only minor modifications. Four different models were trained, each with different encodings of the peptide sequence: (1) sparse matrix encoding, (2) similarity encoding using the Blosum62 matrix, (3) similarity encoding based on the PMBEC matrix³⁴, and (4) an encoding in which each amino acid was represented by the first three principal components derived from dimensionality reduction based on physiochemical properties³⁵. For each model a single hidden layer of 100 neurons with sigmoid activation was used; the outputs of these models were combined in a single output layer to generate the final binding prediction.

For each allele, the positive hits were the MHC-I ligands identified by EpiScan, while the set of negative decoys comprised all other peptides which were identified in the input 9-mer random library but which were not found in any of the EpiScan sorting bins. Training was performed as described⁴, except that a 10-fold excess of decoys was used. Predictive power was assessed as recommended⁴, whereby the ability of the model to predict true binders amongst the top 0.1% of the dataset was evaluated in the presence of a 999-fold excess of decoy peptides (PPV metric). The data depicted in FIG. 3F represents the mean PPV obtained from each of 30 iterations of a five-fold cross-validation procedure (grey dots); for comparison, the mean PPV metric reported for the equivalent allele-specific MSi model for 9-mer peptides (Table S5 of ref⁴) is represented by the black squares.

Conservation Scoring

SARS-CoV-2 protein sequences were obtained from UniProt and entered into the ConSurf Server^{26,27,36}. For S, 3a and 7a RCSB PDB structures (6VXX, 6XDC and 6W37, respectively) were used. HMIVMER was used as the homolog search algorithm with Uniprot as the protein database. Automatic homologue selection settings of a 35-95% homologue identity were required. The alignment method was MAFFT-L-INS-I with Bayesian calculation method with the default evolutionary substitution model. ORF10 was excluded due to lack of a sufficient number of homologues to perform conservation scoring. To locate epitopes in conserved regions, the conservation score was averaged over the length of the epitope.

T Cell Isolation and Expansion

Peripheral blood was provided by collaborators from Ragon Institute of MGH that were PCR-confirmed COVID-19 cases. All study participants provided verbal and/or written informed consent. Participation in these studies was voluntary and the study protocols have been approved by the Partners Institutional Review Board. Memory CD8⁺ T cells were isolated using the Miltenyi CD8⁺ Memory T cell isolation kit according to manufacturer's instructions. T cells were expanded using irradiated peripheral blood mononuclear cells (PBMCs). Briefly, apheresis collars were obtained from the Brigham and Women's Hospital Specimen Bank under protocol T0276 and PBMCs were purified on a Ficoll gradient. The cells at the interface were extracted, washed twice, and irradiated (60 Gy IR). For expansion, isolated memory CD8⁺ patient T cells were added to 2 million irradiated PBMCs in a final volume of 20 ml RPMI, 10% FBS, 100 units/ml penicillin, 0.1 mg/ml streptomycin, 50 U/ml IL-2 (Sigma), and 0.1 ug/ml anti-CD3 antibody (OKT3, ebioscience).

Tetramer Staining of Patient Samples

The following peptides were synthesized by New England Peptide:

| PEPTIDE SEQUNCE | SEQ ID NO: |
|---|---|
| VLYQDVNCTEV | 23 |
| VMVELVAEL | 24 |
| YIDIGNYTV | 25 |
| SLPGVFCGV | 26 |
| NLIDSYFVV | 27 |
| VMAYITGGV | 28 |
| VMAYITGGVV | 29 |
| AMDEFIERYKL | 30 |
| TLIGDCATV | 31 |
| TLATHGLAAV | 32 |

Peptides were loaded at 10 mg/ml and exchange was quantified onto the QuickSwitch Quant HLA-A*02:01 Tetramers (PE or APC labeled) (MBL International) according to manufacturer's instructions. Tetramers were used for staining at a final concentration of 10 μg/ml. Where specified, cells were additionally stained with a Brilliant Violet 421-conjugated anti-CD3 antibody (BioLegend) and an Alexa Fluor 647-conjugated anti-CD8 antibody (Biolegend).

Statistical Tests

Unless otherwise noted, significance for all dot plots was measured by one-way ANOVA with Dunnett's multiple-comparison test with *p<0.05 **p<0.01 ***p<0.001 or ****p<0.0001 for each group relative to the negative control conditions. This was performed using GraphPad Prism 8. Fisher's Exact Test was performed with fishertest using MATLAB R2019b.

Graph Generation

Unless otherwise noted, all dot plots or bar graphs were created using either GraphPad Prism 8 or the Python Seaborn library. Data are represented as mean±SEM of the fold change in mean fluorescence intensity (MFI) relative to the average of the negative controls for that experiment. Each dot represents a different biological replicate. Scatter plots were created using Spotfire 10 (TIBCO).

Logoplot Generation

Logoplots were generated with Seq2Logo³⁷. Logoplots were of type Shannon (-I 1), with Hobohm clustering (-C 2) and no weight on prior (-b 0). To account for the difference in amino acid frequencies between the 9-mer randomer library and the human proteome, for plots describing EpiScan data a custom (--bg argument) position-specific scoring matrix (PSSM) was employed.

Allele Specificity Correlation

For each allele for each methodology, the frequency of every amino acid at each of nine positions was calculated to create a 9×20 matrix. The matrix was flattened into a 1D array and then pairwise Pearson calculations were computed using numpy.corrcoef.

MHC Class I IP Procedure:

1. Cell pellets were thawed on ice, then lysed at 50 million cells/mL of lysis buffer, incubated 30 min on ice

2. Insoluble material was pelleted at 800×g for 5 min.

3. Supernatant was centrifuged at 20,000×g for 30 min at 4° C.

4. Resin was washed and combined with clarified lysates
a. *Saved 200 μL from lysate for ELISA (pre-IP) and BCA.

5. Resin was mixed with lysates (normalized by BCA to lowest protein yield) by gentle rotation at 4° C. overnight.

6. The next day, samples were centrifuged at 800×g for 5 min at 4° C.
b. *Supernatant was reserved for post-IP ELISA

7. Three washes (Buffers 1-3) of the resin were performed, which consisted of the following:
c. Add 2.5 mL of buffer to resin, vortex
d. centrifuge 800×g, 5 min at 4° C.
e. Discard the supernatant.

8. At wash #4, 0.75 mL of Buffer 4 was added, and the total volume was transferred to loBind tubes
f. centrifuge 800×g, 5 min at 4° C.
g. Discard the supernatant.

9. 1 mL of Elution buffer was added to each tube and incubated at 37° C. for 5 min.

10. Samples were centrifuged at 800×g for 5 min at 4° C. to elute.

11. Eluates (supernatant) were collected into new loBind Eppendorf tubes and stored at −80° C. until transfer to MSB.

12. Eluates were submitted for LC-MS/MS analysis and PRE and POST samples were tested by ELISA.

Peptides were desalted and concentrated using a Waters HLB solid phase extraction plate.

Mass Spectrometry

Half of each enriched sample was analyzed by nano LC-MS/MS using a Waters M-Class HPLC system interfaced to a ThermoFisher Fusion Lumos mass spectrometer.

Peptides were loaded on a trapping column and eluted over a 75 μm analytical column at 350 nL/min; both columns were packed with Luna C18 resin (Phenomenex). A 2 hr gradient was employed. The mass spectrometer was operated using a custom data-dependent method, with MS performed in the Orbitrap at 60,000 FWHM resolution and sequential MS/MS performed using high resolution CID and EThcD in the Orbitrap at 15,000 FWHM resolution. All MS data were acquired from m/z 300-800. A 3s cycle time was employed for all steps.

Data Processing

Data were searched using a local copy of PEAKS (Bioinformatics Solutions) with the following parameters:

Enzyme: None

Database: SwissProt Human appended with #1 Bruno_sample 1 or #2 Bruno_sample 2

Fixed modification: None

Variable modifications: Variable modifications: Oxidation (M), Deamidation (N,Q), Acetyl (Protein N-term)

Mass values: Monoisotopic

Peptide Mass Tolerance: 10 ppm

Fragment Mass Tolerance: 0.02 Da

PSM FDR: 1%

PEAKS output was further processed using Microsoft Excel.

### Example 1. Development of EpiScan

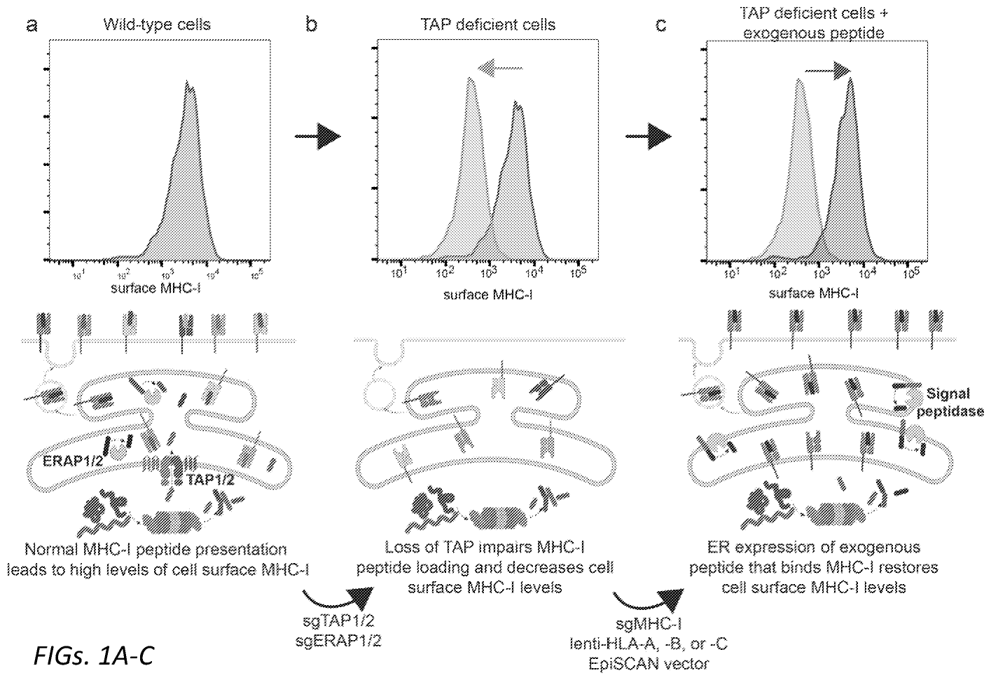

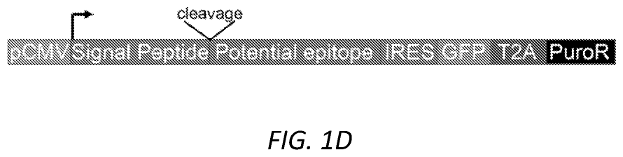

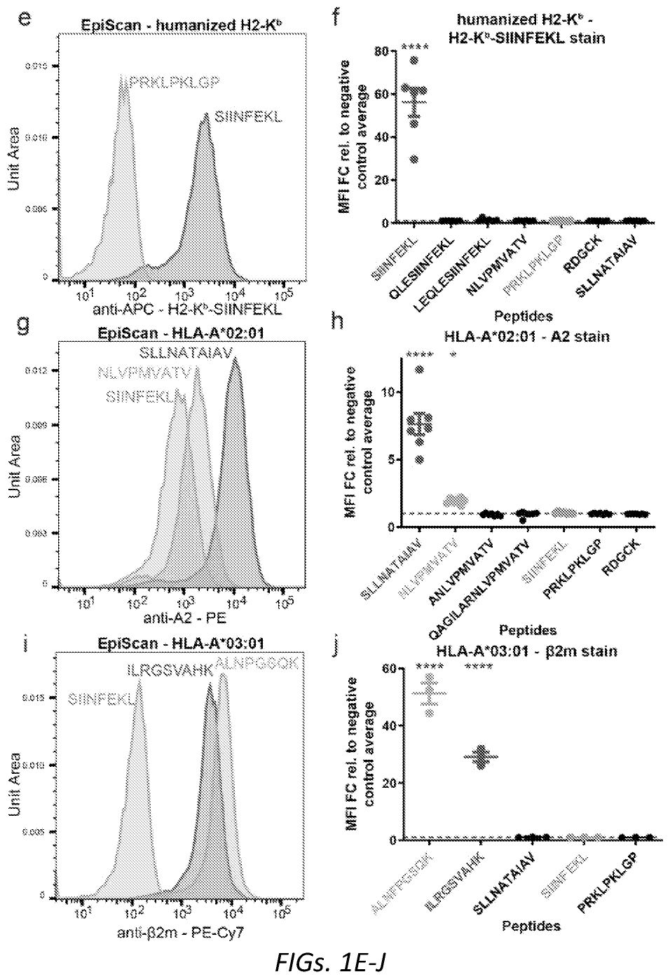

EpiScan is a genetic platform that allows for the high-throughput and cost-efficient identification of peptides that bind MHC-I molecules from within a defined starting pool. EpiScan relies on the principle that MHC-I molecules are only trafficked to, and maintained on, the cell surface after stably binding a high-affinity peptide in the endoplasmic reticulum (ER) (FIG. 1A). In the absence of the TAP complex, which pumps proteasomally-derived peptide fragments into the ER lumen⁵, peptide loading onto MHC-I molecules is impaired and cell surface MHC-I levels are markedly reduced (FIG. 1B). Under these conditions, it was hypothesized that the introduction of a single exogenous high-affinity MHC-I peptide ligand into the ER should restore cell surface MHC-I levels, thereby permitting the binding of individual peptides to MHC-I molecules to be assayed by flow cytometry (FIGS. 1C-D).

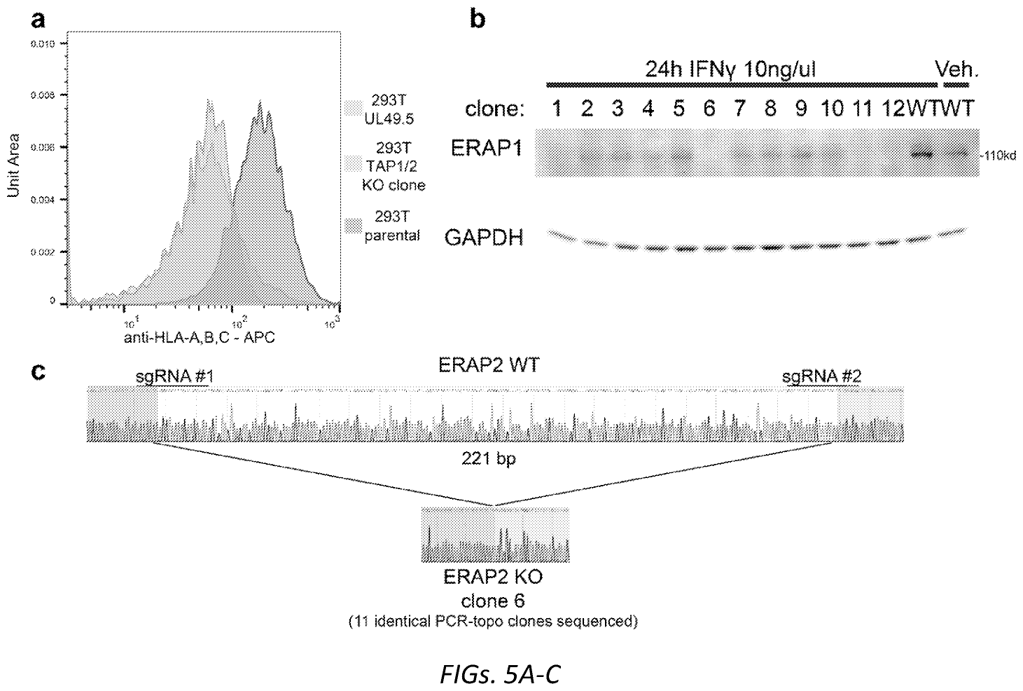

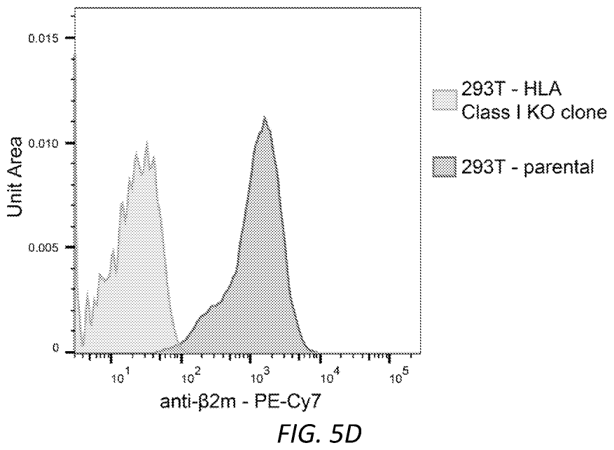

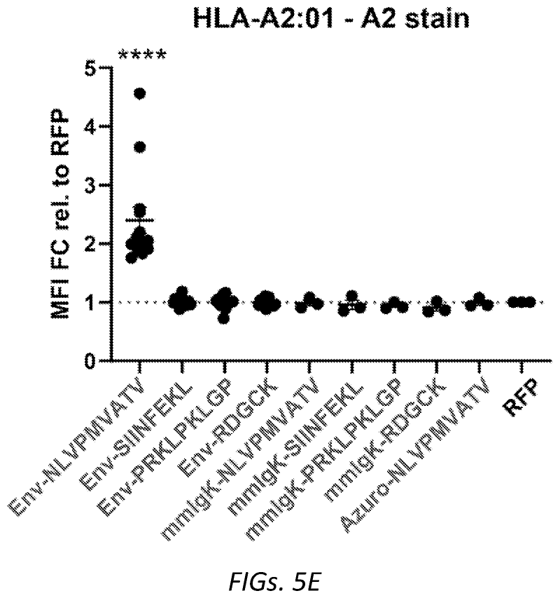

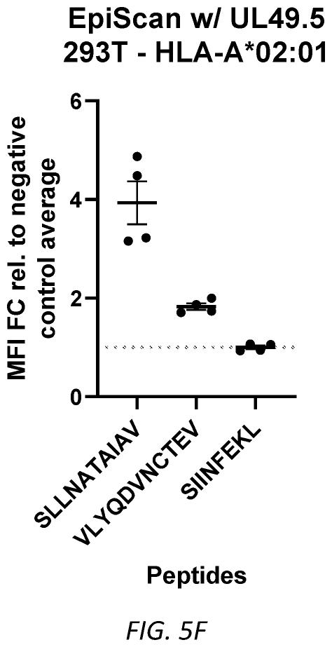

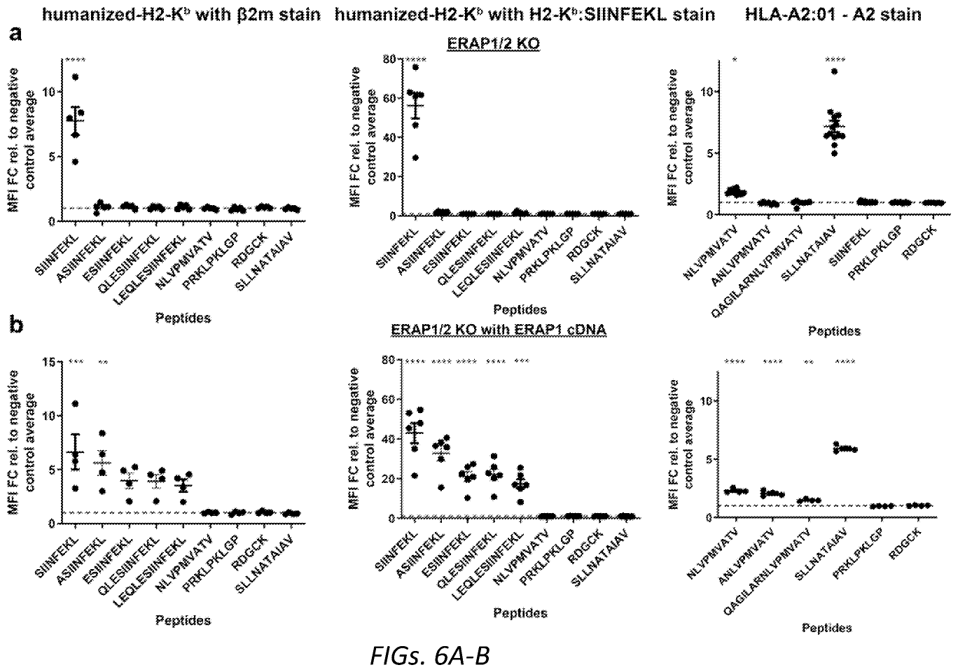

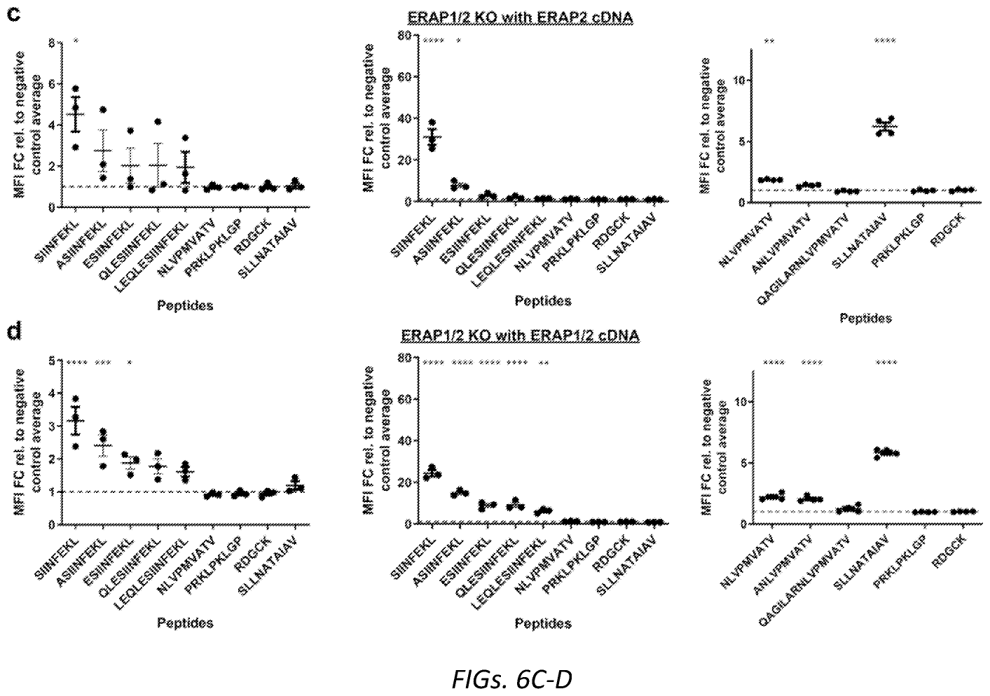

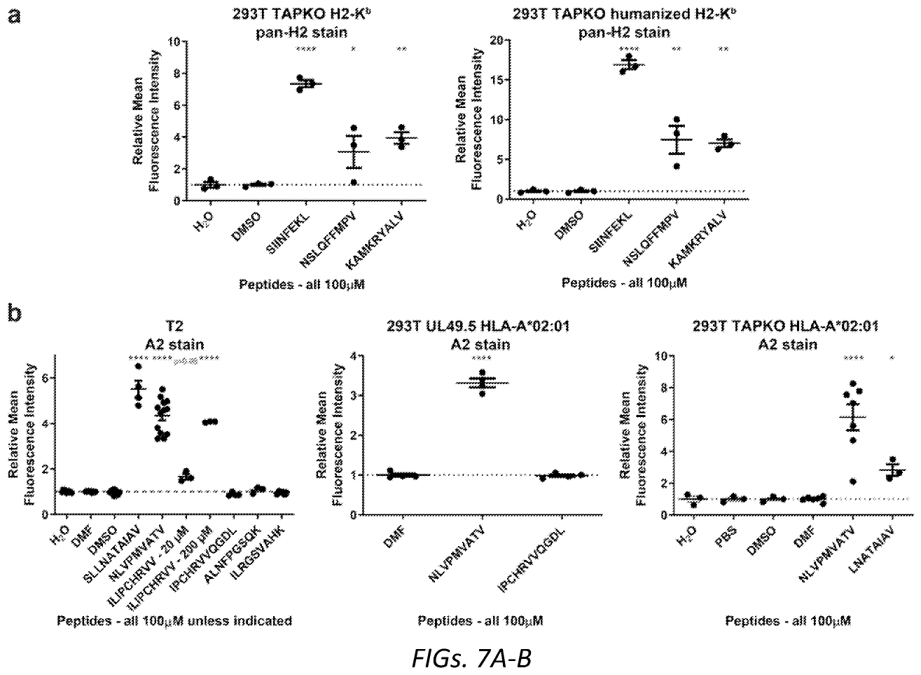

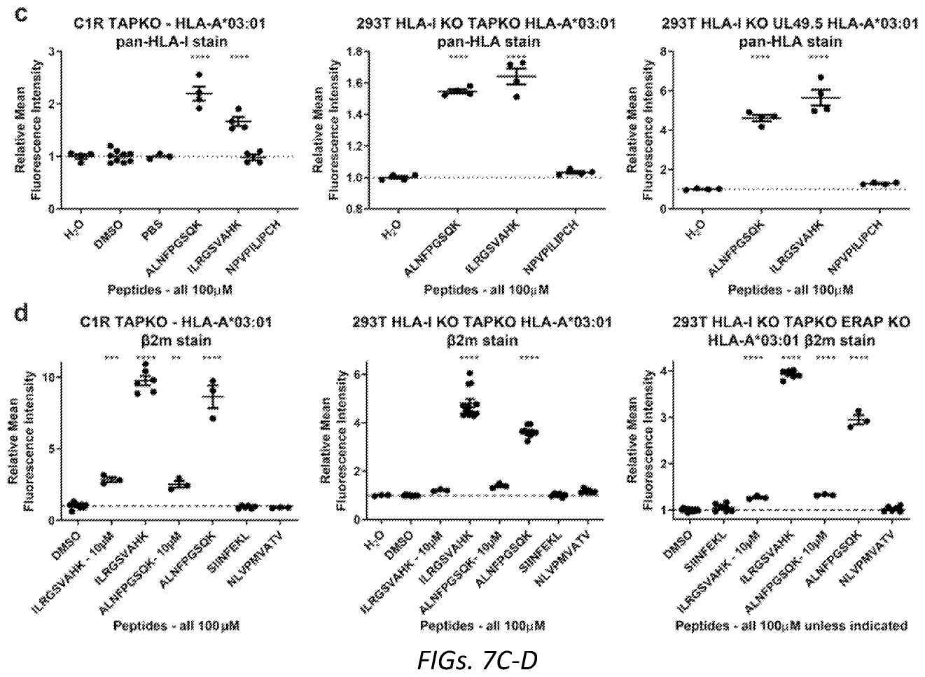

We validated the EpiScan platform using the model ovalbumin antigen, SIINFEKL (SEQ ID NO:33). Using a viral TAP inhibitor gene, UL49.5 (5A) or CRISPR/Cas9-mediated gene disruption, we isolated a HEK 293T clone (henceforth ‘EpiScan cells’) lacking MHC-I (HLA-A, -B, -C), TAP, and the ER-resident metallopeptidases ERAP1 and ERAP2^{6,7} (FIGS. 5B-D). We subsequently re-expressed a single MHC-I allele, a humanized version of the murine H2-K^{b} wherein the beta-2-microglobulin (β2M) interacting domain was replaced with the human equivalent, and examined whether exogenous delivery of the SIINFEKL (SEQ ID NO:33) peptide into the ER would restore cell surface MHC-I levels. Using an expression construct containing the signal peptide from the gp70 gene of mouse mammary tumour virus⁸, we found that exogenous expression of SIINFEKL (SEQ ID NO:33), but not a variety of control peptides, increased cell surface MHC-I levels (FIGS. 1E-F, 5E-F, and FIG. 6A)⁹. In addition, we obtained similar results using the common human MHC-I alleles HLA-A2 and HLA-A3 with corresponding positive control peptides (FIGS. 1G-J). Furthermore, all of the EpiScan results were consistent with peptide pulsing experiments in TAP-deficient cells¹⁰ (FIGS. 7A-D). This shows that synthesized peptides can be used with the EpiScan cells to determine MHC-I binding—they don't have to be genetically encoded.

Peptidase activity in the ER could adversely affect the performance of EpiScan: destruction of the exogenous peptide would reduce the sensitivity of the assay, while partial proteolysis could generate false positives as a processed form of the peptide—and not the genetically-encoded peptide itself—might bind to MHC-I. Thus we also chose to mutate the peptidases ERAP1 and ERAP2, which trim antigenic peptides from their N-termini to generate fragments of the optimal size for MHC-I binding (8-12-mers)^{6,7}. To verify the loss of the activity of these enzymes in EpiScan cells we expressed N-terminally extended versions of our positive control peptides, reasoning that this should not result in increased surface MHC-I levels in the absence of N-terminal peptidase activity. Indeed, N-terminally extended versions of SIINFEKL (SEQ ID NO:33) or NLVPMVATV (SEQ ID NO:34), a peptide derived from the pp65 gene of human cytomegalovirus, did not lead to increased MHC-I surface staining in either humanized-H2-K^{b}- or HLA-A2-expressing EpiScan cells (FIG. 6A). This effect was indeed due to a lack of ERAP1/2 activity, as genetic complementation with exogenous ERAP1 or ERAP2 led to a restoration of cell surface MHC-I levels upon expression of the N-terminally extended peptides (FIGS. 6A-D). Altogether, these data demonstrate that EpiScan constitutes an accurate and robust system for the identification of high-affinity MHC-I peptide ligands. EpiScan thus can be used to determine the effects on peptide presentation of genetic alterations introduced into the cell (see also Example 2, in which effects of a small molecule, abacavir, are evaluated on peptide binding).

### Example 2. High-Throughput MHC-I Ligand Discovery Using EpiScan

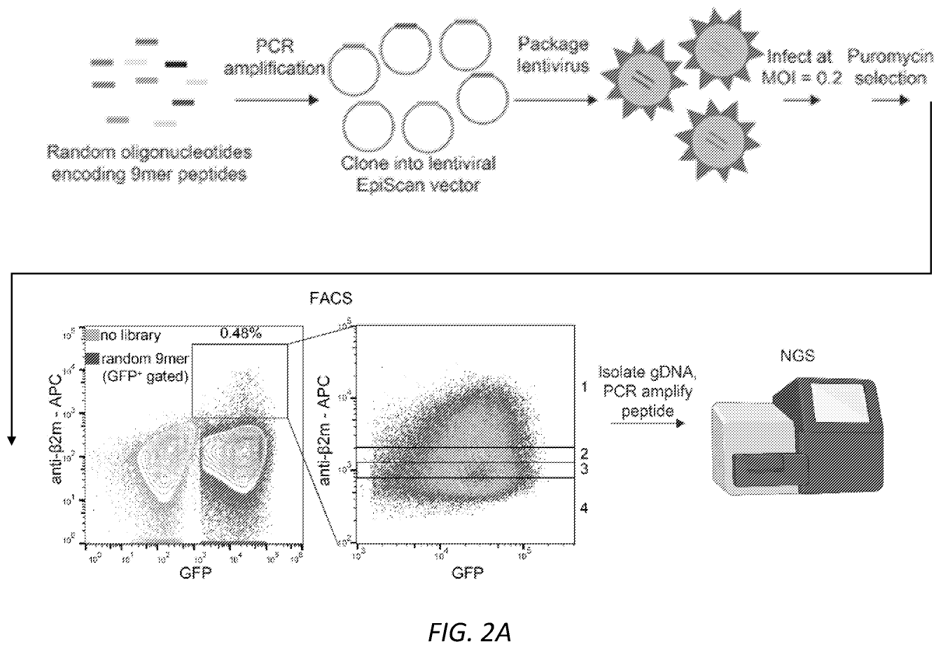

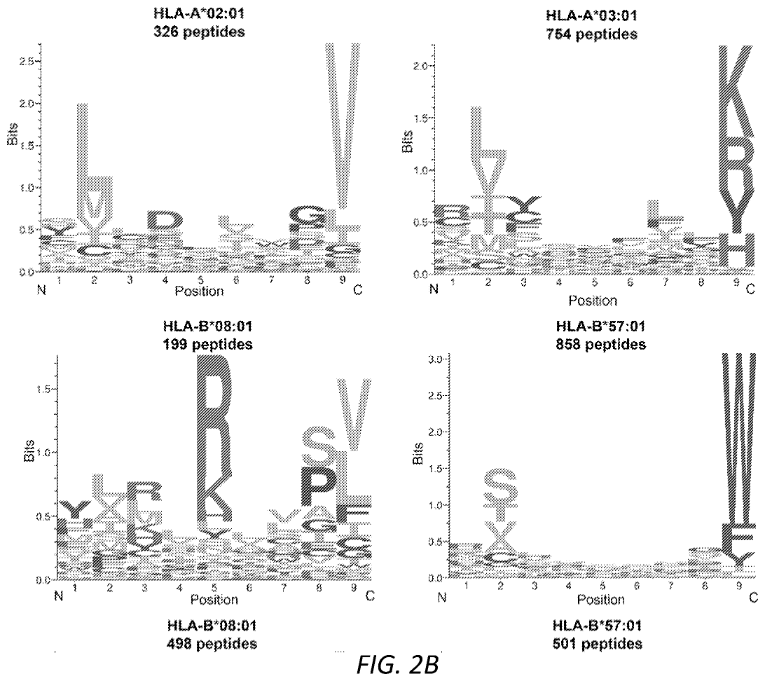

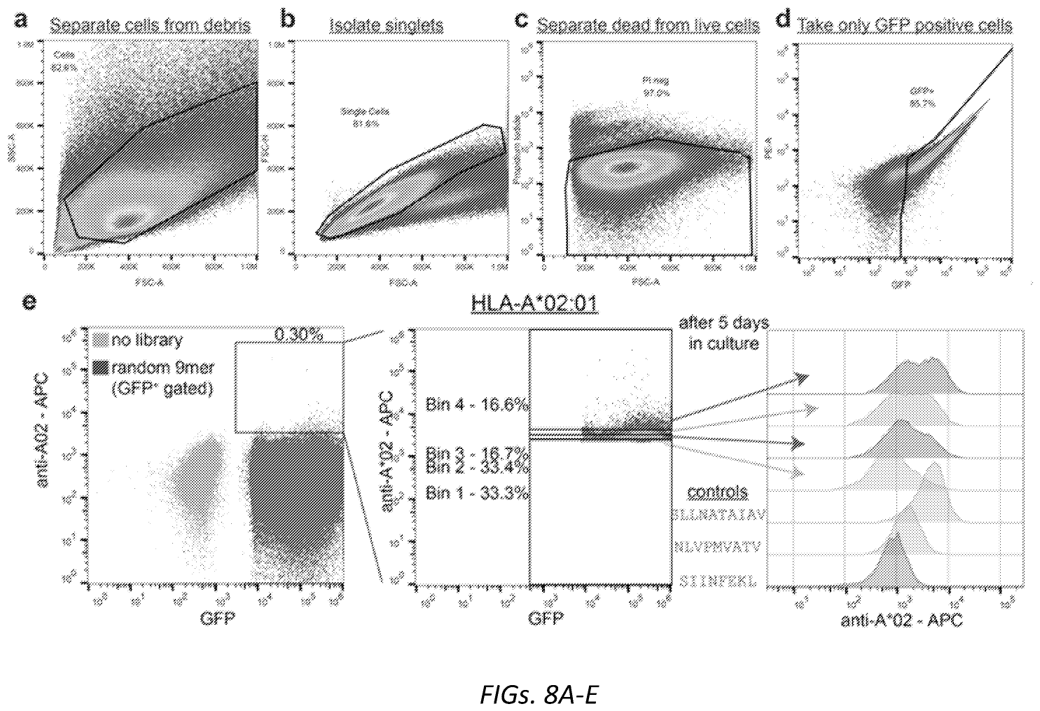

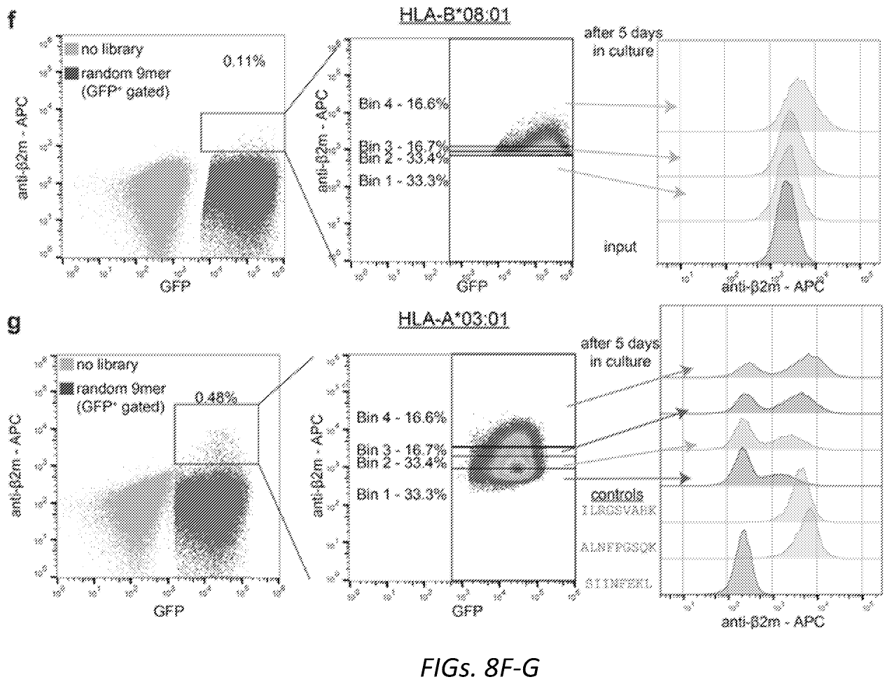

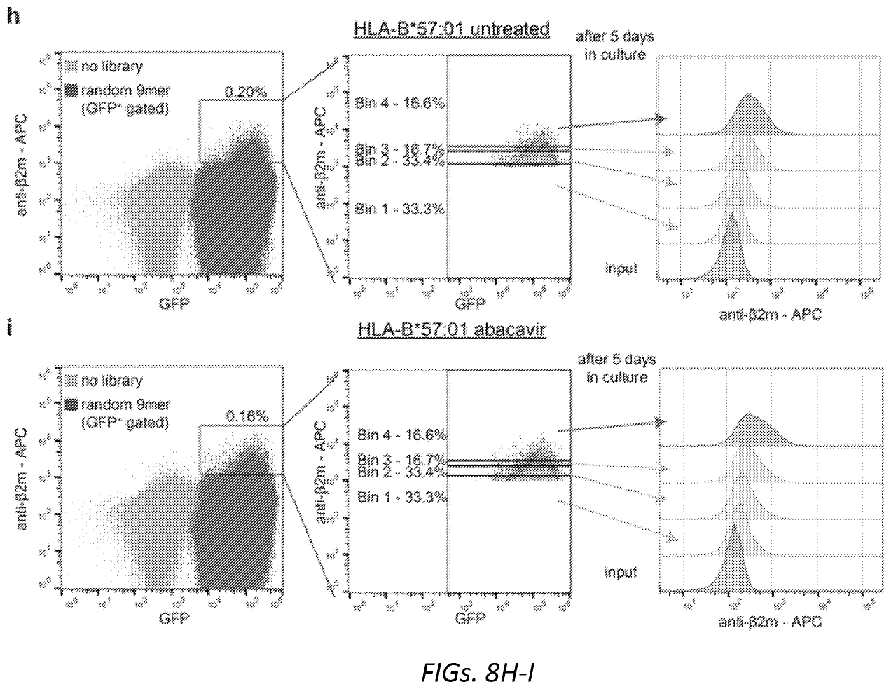

Having optimized the EpiScan platform using individual peptides, we sought to implement the approach for high-throughput screening to identify MHC-I peptide ligands at scale (FIG. 2A). We synthesized a pool of oligonucleotides encoding random 9-mer peptides and cloned them into the EpiScan vector (see FIG. 1D), resulting in a library of ˜500,000 unique 9-mer sequences. The library was packaged into lentiviral particles and introduced into EpiScan cells expressing a single HLA allele at low multiplicity of infection (MOI), such that, following puromycin selection to remove untransduced cells, each cell in the remaining population expressed a single 9-mer peptide. As expected, only a small percentage of these cells exhibited cell surface MHC-I levels above those of the untransduced cells (FIG. 2A, FIGS. 8A-I), consistent with the notion that only a small fraction (˜0.1%) of all possible 9-mer peptides bind any given HLA allele^{11,12}. This positive population was then partitioned into four bins based on the degree of positivity via fluorescence-activated cell sorting (FACS), followed by genomic DNA extraction, PCR amplification of the EpiScan construct, and next-generation sequencing to identify the enriched peptides. We confirmed that the FACS had indeed enriched for cells expressing MHC-I ligands, as, after recovering and expanding, the sorted cells retained elevated surface MHC-I levels (FIGS. 8E-I).

To validate the utility of the EpiScan screening approach, we asked if the sequences of the peptide ligands recapitulated the known preferences of four three common, well studied, MHC-I alleles: HLA-A2, HLA-A3, HLA-B8 and HLA-B57. In each case, the sequences of the high-confidence peptides identified by EpiScan closely mirrored those of the corresponding sequences identified by mass spectrometry⁴ (FIGS. 2B-C). For this analysis, the sorting bins were treated as replicate experiments and high-confidence MHC-I binders were identified based on reproducible enrichments across the four bins (see Methods). All peptides ligands identified by EpiScan were ranked based on the degree to which the distribution of sequencing reads was skewed toward the highest bin; thus, if a peptide had significantly more reads in bin 4 than bin 1, it would receive a higher ranking. Logoplots were generated to compare the sequences of the top 100 or 200 peptides, compared to the bottom 100 or 200 peptides. For HLA-A3, however, a progressive increase in cell surface MHC-I levels was observed across the four bins (FIG. 8G). Future benchmarking against a library of peptides with known affinity will allow us to interpret the relative affinity of different peptides based on the distribution of sequencing reads across the sorting bins.

We further validated our EpiScan screening approach by investigating the underlying causes of abacavir hypersensitivity syndrome. Abacavir is an HIV reverse transcriptase inhibitor that causes hypersensitivity in around 5% of patients¹³; predisposition to abacavir hypersensitivity reactions is strongly associated with HLA*B57:01, and crystal structures show abacavir binding in the peptide binding groove of HLA*B57:01^{14,15}. Screening a library of random 9-mer peptides in HLA-B57-expressing EpiScan cells in the presence and absence of abacavir yielded both overlapping and distinct sets of binding peptides. Consistent with previous mass spectrometry-based studies^{14,15}, the primary difference between the two conditions occurs at the C-terminal anchor position: whereas the two most common anchor residues, tryptophan and phenylalanine, were present at equal frequency in both conditions, the frequency of tyrosine decreased upon abacavir treatment while the frequency of valine and isoleucine increased, as shown in the following table.

| C-terminal Residue | Untreated | Abacavir treated | p-value |
|---|---|---|---|
| V | 0 | 28 | 1.51E−10 |
| I | 24 | 59 | 1.19E−06 |
| Y | 51 | 22 | 0.011 |
| W | 655 | 502 | 0.027 |
| F | 107 | 66 | 0.062 |
| R | 0 | 3 | 0.091 |
| C | 0 | 3 | 0.091 |
| G | 1 | 4 | 0.181 |
| M | 3 | 5 | 0.480 |
| L | 15 | 10 | 0.688 |
| N | 2 | 1 | 1.000 |

This difference would create a significant number of novel peptides displayed by HLA*B57:01 and explains the widespread T cell activation elicited in the hypersensitivity reaction. Thus, EpiScan is capable of detecting subtle changes in MHC-I binding specificity and can be further exploited to investigate autoimmunity and the interactions of drugs with the immune system.

### Example 3. EpiScan and Mass Spectrometry Represent Complementary Approaches for MHC-I Ligand Discovery

Mass spectrometry (MS) represents the current best-in-class method for high-throughput MHC-I immunopeptidomics, and thus we wanted to scrutinize the differences between EpiScan and MS in an unbiased manner. First, we used unsupervised clustering to examine the similarities between the MHC-I ligands identified by MS and EpiScan. The clustering indicated that the differences between alleles was greater than the differences between the two methodologies (FIG. 3A). Additionally, we noticed correlation between HLA-A02 and HLA-B08, and to a lesser extent, HLA-A02 and HLA-A03, suggesting potential for the alleles to share peptide ligands.

For all four MHC-I alleles we noticed modest differences between the peptide binding preferences as determined by EpiScan and MS (FIG. 2B-C). Even after normalizing for the differences in amino acid frequencies in our 9-mer randomer peptide library compared to the human proteome, cysteine was greatly enriched across all peptide positions among the MHC-I peptide ligands identified by EpiScan versus those identified by MS (FIG. 3C), while for HLA-A2 and HLA-B8 proline was highly represented at the penultimate position. Proteasome cleavage is strongly disfavoured downstream of proline residues^{16,17}; thus the position-specific enrichment of proline emphasizes that peptide ligands are detected by EpiScan solely on the basis of MHC-I affinity, whereas the endogenous MHC-I ligands detected by mass spectrometry approaches are impacted by proteasome cleavage preferences. As a result of its varied in vivo modifications and its propensity for oxidation during sample preparation, cysteine-containing peptides are known to be difficult to identify by MS². Indeed, cysteine was present at roughly the expected frequency across the MHC-I ligands detected by EpiScan, but was dramatically depleted across those peptides identified by MS (FIG. 3C). To further validate these findings, we selected a panel of high-confidence HLA-A3 ligands detected by EpiScan that (1) contained cysteine residues and (2) were not predicted to bind by NetMHC4.0 or HLAthena (Table 1)^{4-18,19} and performed individual EpiScan assays: all of the peptides increased surface MHC-I levels at least 20-fold compared to negative controls (FIG. 3D). Thus, we conclude that cysteine-containing peptides are underrepresented in MS-based datasets of MHC-I ligands and that EpiScan represents a complementary technique for the detection of CD8⁺ T cell epitopes.

TABLE 1
HLA-A*03:01 binding predictions for example
cysteine-containing peptides.

| | SEQ ID | NetMHC | | EpiScan | EpiScan |
|---|---|---|---|---|---|
| Peptide | NO: | 4.0 (nM) | MSi | predictor | MFI |
| CLFCEVLVH | 35 | 2985.8 | 0.2244 | 0.9999 | 40.94 |
| RCFQWALMY | 36 | 1467 | 0.5855 | 1 | 19.34 |
| LTCSLLLWH | 37 | 3682.7 | 0.4304 | 0.9985 | 30.60 |
| RLCSDVWLH | 38 | 2387.3 | 0.4339 | 0.9966 | 48.14 |
| MTCARVLCH | 39 | 1546.3 | 0.1032 | 1 | 44.26 |
| TVSSIILRH | 40 | 1751.7 | 0.9653 | 0.9853 | 51.53 |
| NIAKFTLSH | 41 | 2700.4 | 0.5899 | 0.9915 | 30.95 |

An important goal in the field of immunopeptidomics is the development of computational models that can accurately predict MHC-I ligands starting from the primary sequence of a protein^{4,20,21}. Given the differences between the MHC-I ligands identified by EpiScan and MS, we wanted to provide proof-of-principle that an effective prediction algorithm could be developed from EpiScan data. Using a neural network architecture analogous to the MSi algorithm recently developed by Sarkizova and colleagues⁴ (FIG. 3E), we developed EpiScan Predictor, or ESP. We trained machine learning models to classify 9-mer peptide sequences as binders or non-binders for HLA-A2, HLA-A3, HLA-B8 and HLA-B57. As proposed previously^{4,17}, we evaluated the positive predictive value (PPV) of these models based on their ability to correctly identify true binders (peptide ligands identified in the random 9-mer EpiScan screens) in the presence of a 999-fold excess of random decoys. Overall, the performance of our ESP models was roughly comparable to the MSi models⁴ (FIG. 3F), and, when used to predict 9-mer MHC-I ligands across the entire human proteome, MHC-I binders predicted by ESP but not by MSi reflected the differences in amino acid composition discussed above, including the enrichment of cysteine and proline. The predictive power of ESP could be significantly improved by screening focused pools of peptides that would provide a larger volume of more informative training data. In addition to not suffering from detection bias inherent to MS, ESP renders predictions solely based on allele-specific affinity, and thus can identify MHC-I ligands that aren't subject to proteasome processing or TAP import.

### Example 4. Targeted Immunopeptidomics: EpiScan Reveals CD8⁺ T Cell Epitopes from SARS-CoV-2

The key advantage of EpiScan over MS-based approaches is that it permits the targeted identification of MHC-I ligands from a defined pool of potential epitopes. The novel coronavirus, SARS-CoV-2, has spread rapidly across the globe; as of early July 2020, SARS-CoV-2 had caused over 12 million confirmed infections and was responsible for over 500,000 deaths. Outcomes resulting from SARS-CoV-2 infection vary greatly for individuals²², and recent work has shown that a robust T cell response is correlated with favourable outcomes²²⁻²⁴. Therefore, we set out to exploit the programmability of EpiScan to perform a comprehensive screen of the SARS-CoV-2 genome for MHC-I ligands.

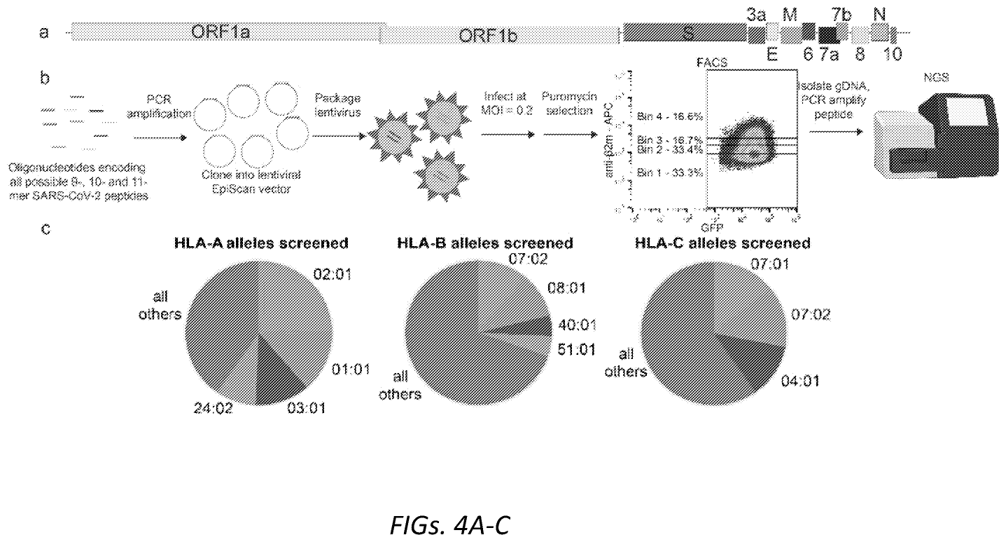

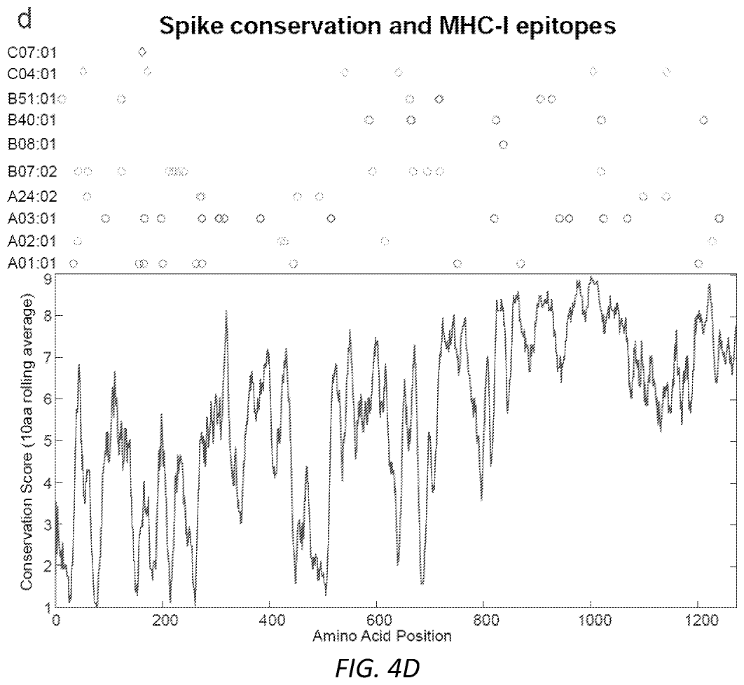

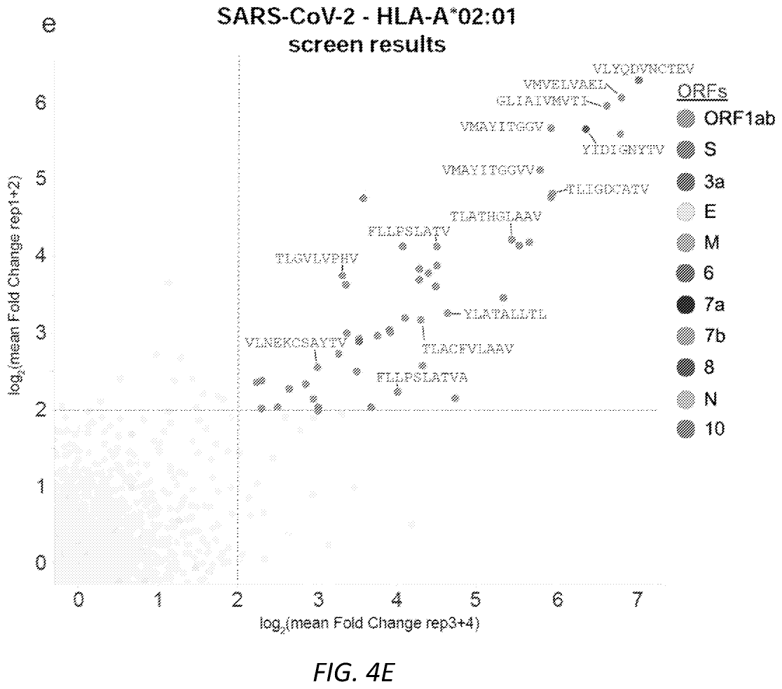

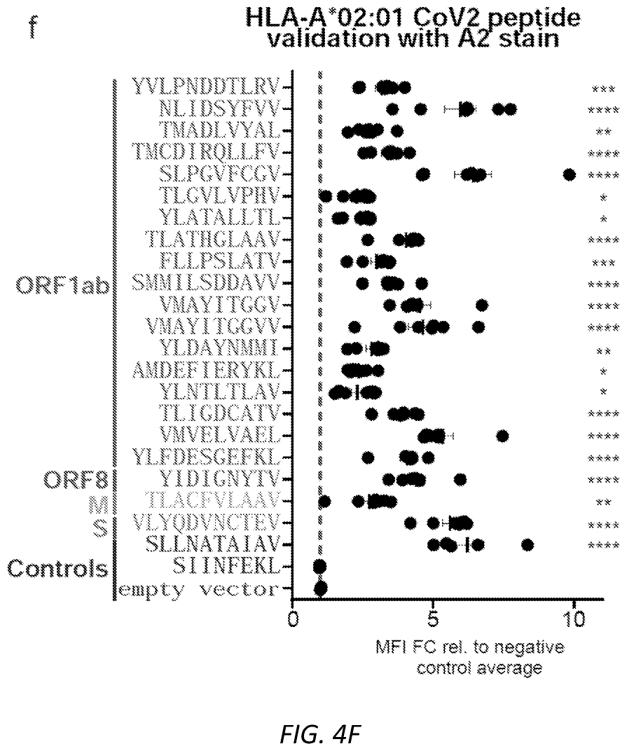

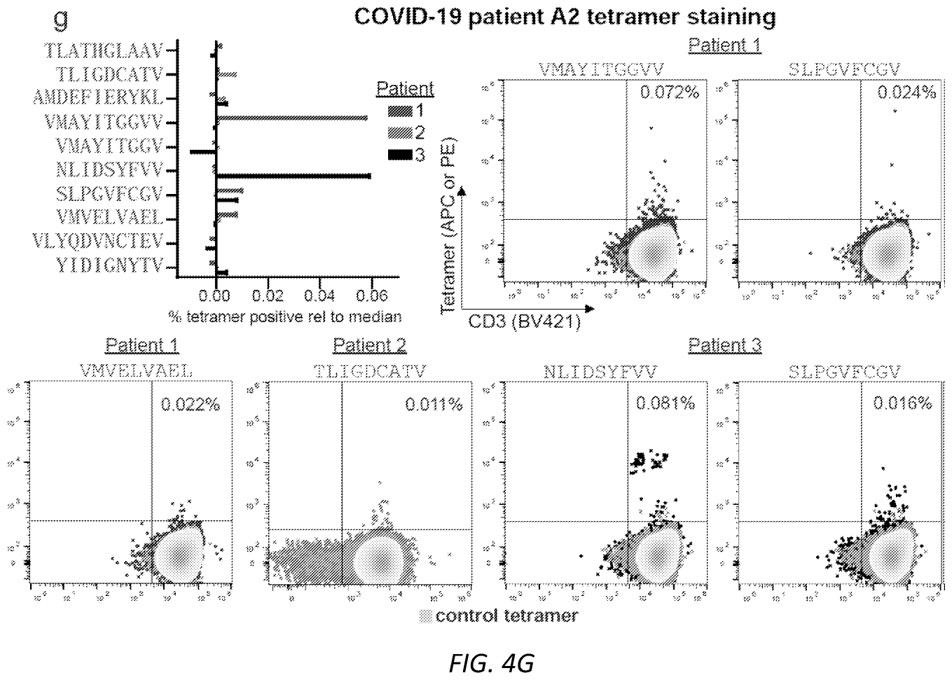

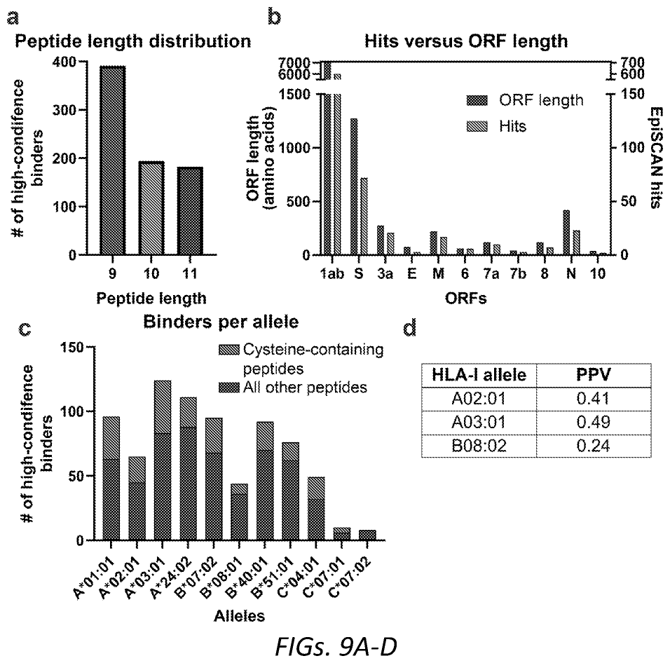

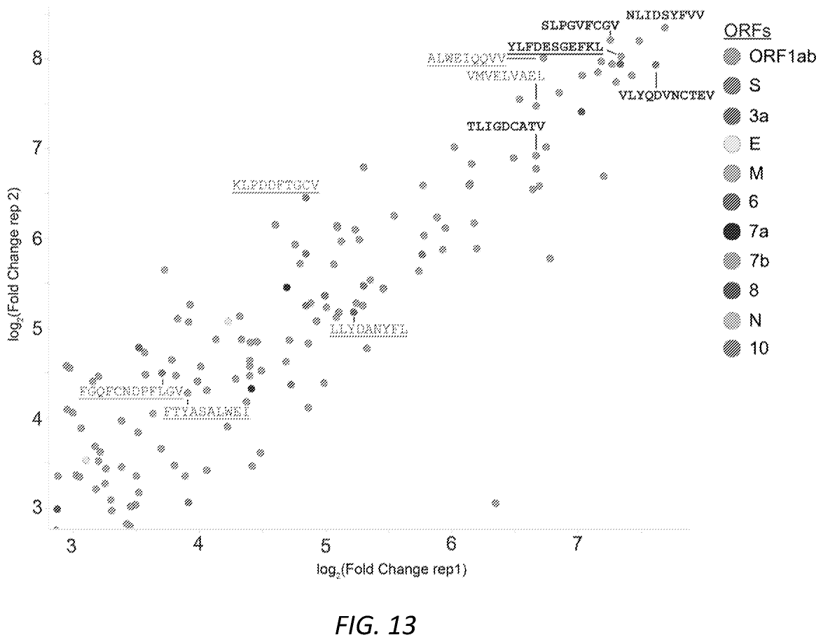

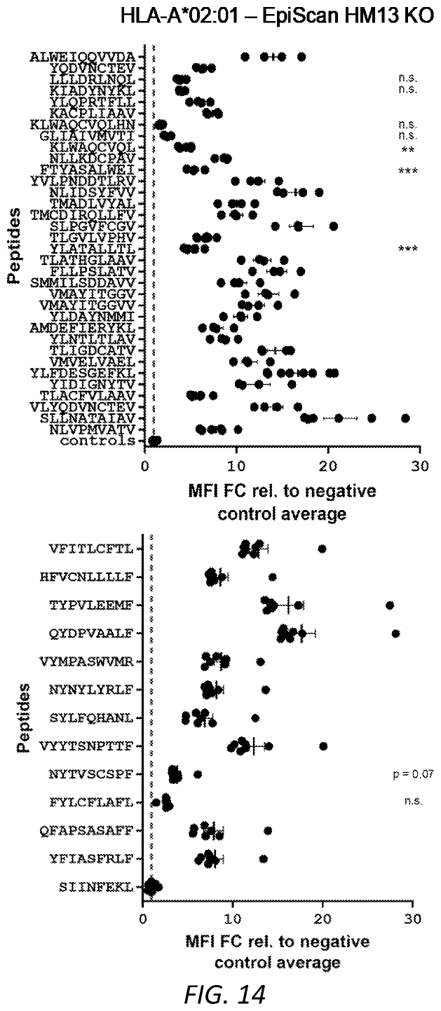

We synthesized an oligonucleotide library encoding all possible 9-, 10- and 11-mer peptides covering 11 different strains of SARS-CoV-2 (a total of ˜30,000 sequences), and performed a series of EpiScan screens using a panel of cell lines expressing 11 of the most common HLA-I alleles (FIG. 4A-C). Additionally, HLA-A*02:01 was screened in EpiScan cells without HM13 (FIG. 13). We identified high-confidence binders for each allele tested from every open reading frame (ORF) of the virus (FIG. 4D, FIGS. 9A-C). The number of hits per ORF increased with the length of the ORF (FIG. 9B). Notably, approximately one-quarter of all ligands identified contained one or more cysteine residues, which would likely have escaped detection by MS-based approaches (FIG. 9C). We found 72 high-confidence binders derived from the spike glycoprotein (S) across 10 of the alleles screened (FIG. 4D), and 65 potential epitopes across the entire virus for HLA-A2 alone (FIG. 4E). Optimal peptides for a potential CD8⁺ T cell vaccine are those that bind more than one HLA allele in order to be efficacious in the largest number of individuals and that are derived from regions that are evolutionarily conserved across coronaviruses to hinder viral escape²⁵: we identified 33 peptides that bound more than one HLA allele (Table 2), and 77 peptides located in highly conserved regions (FIG. 4D and Table 3)^{26,27}. Furthermore, peptides unique to SARS-CoV-2 among the human coronaviruses will be important for assessing T cell-based immunity, particularly in seronegative individuals (Table 2)²⁸. Individual EpiScan experiments validated 100% (21 of 21) of the top candidate ligands for HLA-A2 (FIGS. 4E-F, 14). The results demonstrated that EpiScan SARS-CoV-2 screening successfully identifies peptides that are recognized in the course of the natural immune response to SARS-CoV-2 infection.

Additionally, we used this independent dataset to evaluate the performance of our computational models that were trained on the random 9-mer data; we found that the models had comparable predictive power when applied to the SARS-CoV-2 EpiScan screens (FIG. 9D).

TABLE 2
High-confidence SARS-CoV-2 MHC-I peptide ligands that bind
more than one allele and their uniqueness among common
human coronaviruses.

| | | | | | | | | Unique |
|---|---|---|---|---|---|---|---|---|
| | | | | | | | | to |
| | | | | | | Allele | Allele | SARS- |
| AA seq | # | Length | Uniprot_ID | Protein | Span | 1 | 2 | CoV-2? |
| ATSRTLSYY | 42. | 9 | QHD43419 | M | 171-179 | A01:01 | A03:01 | y |
| KFPRGQGVPI | 43. | 10 | QHD43423 | N | 65-74 | B07:02 | A03:01 | y |
| NPANNAAIV | 44. | 9 | QHD43423 | N | 150-158 | B07:02 | B40:01 | y |
| VPHVGEIPV | 45. | 9 | QHD43415 | Orf1ab | 108-116 | B07:02 | C07:01 | y |
| YPLECIKDL | 46. | 9 | QHD43415 | Orf1ab | 196-204 | B07:02 | B08:01 | y |
| VMAYITGGV | 28. | 9 | QHD43415 | Orf1ab | 597-605 | B51:01 | B07:02 | y |
| YPQVNGLTSI | 47. | 10 | QHD43415 | Orf1ab | 1658-1667 | B51:01 | B07:02 | y |
| LACEDLKPV | 48. | 9 | QHD43415 | Orf1ab | 2039-2047 | A02:01 | B51:01 | y |
| VPMEKLKTL | 49. | 9 | QHD43415 | Orf1ab | 2604-2612 | B07:02 | B51:01 | y |
| VAKSHSIAL | 50. | 9 | QHU36823 | Orf1ab | 2703-2711 | B07:02 | B51:01 | y |
| MPASWVMRI | 51. | 9 | QHD43415 | Orf1ab | 3655-3663 | B51:01 | C07:01 | y |
| KMADQAMTQMY | 52. | 11 | QHD43415 | Orf1ab | 4003-4013 | A03:01 | B07:02 | y |
| CTDDNALAY | 53. | 9 | QHD43415 | Orf1ab | 4163-4171 | A01:01 | B08:01 | y |
| VTANVNALL | 54. | 9 | QHD43415 | Orf1ab | 5092-5100 | A24:02 | A01:01 | y |
| LAIDAYPLTK | 55. | 10 | QHD43415 | Orf1ab | 5254-5263 | A03:01 | B07:02 | y |
| AIDAYPLTK | 56. | 9 | QHD43415 | Orf1ab | 5255-5263 | A03:01 | A01:01 | y |
| TPHTVLQAV | 57. | 9 | QHD43415 | Orf1ab | 5318-5326 | B51:01 | A02:01 | y |
| ALCEKALKY | 58. | 9 | QHD43415 | Orf1ab | 5640-5648 | A01:01 | A03:01 | y |
| LPIDKCSRI | 59. | 9 | QHD43415 | Orf1ab | 5649-5657 | B07:02 | A03:01 | y |
| KSAQCFKMFY | 60. | 10 | QHD43415 | Orf1ab | 5791-5800 | A03:01 | B07:02 | y |
| SPYNSQNAV | 61. | 9 | QHD43415 | Orf1ab | 5837-5845 | B07:02 | A03:01 | y |
| TVDSSQGSEY | 62. | 10 | QHD43415 | Orf1ab | 5856-5865 | A01:01 | A03:01 | n |
| IPLMYKGLL | 63. | 9 | BBW89516 | Orf1ab | 6067-6075 | B07:02 | C04:01 | y |
| TYACWHHSIGF | 64. | 11 | QHD43415 | Orf1ab | 6148-6158 | B08:01 | B07:02 | y |
| DAIMTRCLAV | 65. | 10 | QHD43415 | Orf1ab | 6198-6207 | B08:01 | B08:01 | n |
| KRVDWTIEY | 66. | 9 | QHD43415 | Orf1ab | 6213-6221 | C07:01 | B51:01 | y |
| VPLKSATCI | 67. | 9 | QHD43415 | Orf1ab | 6391-6399 | B51:01 | B07:02 | n |
| AMDEFIERYKL | 30. | 11 | QHD43415 | Orf1ab | 6669-6679 | B40:01 | B07:02 | y |
| IMRTFKVSI | 68. | 9 | QHD43420 | 6 | 18-26 | B51:01 | B07:02 | y |
| IIKNLSKSL | 69. | 9 | QHD43420 | 6 | 36-44 | B07:02 | B51:01 | y |
| IPYNSVTSSI | 70. | 10 | QHD43417 | 3a | 158-167 | B07:02 | B51:01 | y |
| IPYNSVTSSIV | 71. | 11 | QHD43417 | 3a | 158-168 | B51:01 | B07:02 | y |
| IVNNATNVV | 72. | 9 | QHD43416 | S | 119-127 | B51:01 | A24:02 | y |
| SANNCTFEY | 73. | 9 | QHD43416 | S | 162-170 | A03:01 | B51:01 | y |
| IPTNFTISV | 74. | 9 | QHD43416 | S | 714-722 | B07:02 | A24:02 | y |
| VYDPLQPEL | 75. | 9 | QHD43416 | S | 1137-1145 | C04:01 | B07:02 | y |

#, SEQ ID NO:

TABLE 3
SARS-CoV-2 MHC-I peptide ligands located in
regions of high sequence conservation. The
conservation score (determined by ConSurf)
was averaged over the length of the peptide
and those with a score over 7.85 were
selected, so as to capture ~10% of the total
high-confidence binders.

| | | SEQ | | |
|---|---|---|---|---|
| ORF | peptide | ID NO: | allele | score |
| 7a | TLATCELYH | 76. | A03 | 8.50 |
| N | SWFTALTQH | 77. | B07 | 7.89 |
| ORF1ab | TMCDIRQLLF | 78. | A24 | 8.20 |
| ORF1ab | VYIGDPAQL | 79. | A24 | 9.00 |
| ORF1ab | YYSLLMPIL | 80. | A24 | 7.89 |
| ORF1ab | KYTQLCQYL | 81. | A24 | 8.78 |
| ORF1ab | VFVLWAHGF | 82. | A24 | 7.89 |
| ORF1ab | YYSLLMPILTL | 83. | A24 | 7.91 |
| ORF1ab | YFIKGLNNL | 84. | A24 | 8.33 |
| ORF1ab | TVDSSQGSEY | 85. | A01 | 8.90 |
| ORF1ab | AIDAYPLTK | 86. | A01 | 8.78 |
| ORF1ab | IVDTVSALVY | 87. | A01 | 8.00 |
| ORF1ab | MADQAMTQMY | 88. | A01 | 8.00 |
| ORF1ab | VTDVTQLYL | 89. | A01 | 8.22 |
| ORF1ab | ATEETFKLSY | 90. | A01 | 8.00 |
| ORF1ab | LAIDAYPLTK | 91. | A01 | 8.80 |
| ORF1ab | ESFGGASCCLY | 92. | A01 | 8.45 |
| ORF1ab | AIDAYPLTKHP | 93. | A01 | 8.09 |
| ORF1ab | KATEETFKLSY | 94. | A01 | 8.09 |
| ORF1ab | KMADQAMTQMY | 95. | A01 | 8.00 |
| ORF1ab | SMMILSDDAVV | 96. | A02 | 8.91 |
| ORF1ab | YLNTLTLAV | 97. | A02 | 8.00 |
| ORF1ab | TMCDIRQLLFV | 98. | A02 | 8.00 |
| ORF1ab | TMADLVYAL | 99. | A02 | 8.11 |
| ORF1ab | RLANECAQV | 100. | A02 | 8.78 |
| ORF1ab | VQQWGFTGNLQ | 101. | A02 | 8.27 |
| ORF1ab | ELPTGVHAG | 102. | A02 | 7.89 |
| ORF1ab | KCTSVVLLSV | 103. | A02 | 8.30 |
| ORF1ab | IMASLVLAR | 104. | A03 | 8.00 |
| ORF1ab | IMASLVLARK | 105. | A03 | 8.10 |
| ORF1ab | RIMASLVLARK | 106. | A03 | 8.18 |
| ORF1ab | AIDAYPLTK | 107. | A03 | 8.78 |
| ORF1ab | QTMLFTMLRK | 108. | A03 | 8.00 |
| ORF1ab | TMLFTMLRK | 109. | A03 | 7.89 |
| ORF1ab | VLHDIGNPK | 110. | A03 | 8.11 |
| ORF1ab | MADQAMTQMYK | 111. | A03 | 8.09 |
| ORF1ab | SICSTMTNR | 112. | A03 | 8.78 |
| ORF1ab | LAIDAYPLTK | 113. | A03 | 8.80 |
| ORF1ab | KMADQAMTQMY | 114. | A03 | 8.00 |
| ORF1ab | MASLVLARK | 115. | A03 | 8.00 |
| ORF1ab | MTNRQFHQK | 116. | A03 | 8.67 |
| ORF1ab | RQFHQKLLK | 117. | A03 | 8.22 |
| ORF1ab | ATVVIGTSK | 118. | A03 | 8.33 |
| ORF1ab | DAIMTRCLAV | 119. | B07 | 8.20 |
| ORF1ab | MPNMLRIMASL | 120. | B07 | 8.27 |
| ORF1ab | APRTLLTKGTL | 121. | B07 | 8.18 |
| ORF1ab | MPNMLRIMA | 122. | B07 | 8.11 |
| ORF1ab | IPLMYKGLL | 123. | B07 | 8.00 |
| ORF1ab | SPYNSQNAV | 124. | B07 | 8.67 |
| ORF1ab | LPVNVAFEL | 125. | B07 | 8.78 |
| ORF1ab | SARIVYTAC | 126. | B07 | 8.11 |
| ORF1ab | ICQAVTANV | 127. | B07 | 8.56 |
| ORF1ab | VCRFDTRVL | 128. | B07 | 8.33 |
| ORF1ab | ITRAKVGIL | 129. | B07 | 8.33 |
| ORF1ab | LMIERFVSL | 130. | B08 | 8.22 |
| ORF1ab | YLRKHFSMMIL | 131. | B08 | 8.45 |
| ORF1ab | YLRKHFSMM | 132. | B08 | 8.33 |
| ORF1ab | DAIMTRCLAV | 133. | B08 | 8.20 |
| ORF1ab | TERLKLFAA | 134. | B08 | 8.22 |
| ORF1ab | TAYANSVFNI | 135. | B51 | 9.00 |
| ORF1ab | FPLCANGQV | 136. | B51 | 8.33 |
| ORF1ab | SPYNSQNAV | 137. | B51 | 8.67 |
| ORF1ab | VPYNMRVIH | 138. | B51 | 8.44 |
| ORF1ab | TVDSSQGSEY | 139. | B51 | 8.90 |
| ORF1ab | IPLMYKGLL | 140. | C41 | 8.00 |
| ORF1ab | WAHGFELTS | 141. | C41 | 8.44 |
| ORF1ab | VNVAFELWAKR | 142. | C41 | 8.36 |
| ORF1ab | VVFDEISMATN | 143. | 071 | 8.64 |
| S | LIDLQELGKY | 144. | A01 | 7.90 |
| S | AQALNTLVK | 145. | A03 | 7.89 |
| S | RSFIEDLLFNK | 146. | A03 | 8.36 |
| S | GIYQTSNFR | 147. | A03 | 8.44 |
| S | AEIRASANL | 148. | B40 | 7.89 |
| S | IEDLLFNKVTL | 149. | B40 | 8.09 |
| S | IANQFNSAI | 150. | B51 | 8.33 |
| S | MAYRFNGIGV | 151. | B51 | 8.10 |
| S | RLQSLQTYVT | 152. | C41 | 8.70 |

Lastly, we evaluated whether COVID-19 patients mount T cell responses against these epitopes. For 10 of the validated HLA-A2 ligands, we generated peptide-MHC tetramers (Table 4) and used them to assess the prevalence of reactive CD8⁺ T cells in the blood of convalescent COVID-19 patients. Each of the three patients tested had CD8⁺ T cells that reacted with at least one of the 10 tetramers (FIG. 4G). Importantly, one of these peptides, VMAYITGGVV (SEQ ID NO:29), was not predicted to bind by NetMHC4.0 or HLAthena (Table 4). Although our approach is agnostic to immune responses and only evaluates peptide affinity for MHC-I, our data support the notion that T cell responses are enriched for high affinity peptide:MHC-I interactions²⁹. Our implementation of EpiScan to identify MHC-I ligands from SARS-CoV-2 represents the first effort to experimentally query all the potential CD8⁺ T cell epitopes from a single organism in a systematic way.

TABLE 4
Binding predictions for SARS-CoV-2 HLA-A*02:01 peptides
used for tetramer staining. MSi and ESP predictions
are represented as a probability of being a binder, thus
a score closer to 1 is more like to be a binder. N/A for
ESP indicates that no predictions could be made
because to date the models are only trained on 9mers.
Column second-from-right is quantification of QuickSwitch
Quant HLA-A*02:01 Tetramer control peptide exchange.
Patient tetramer positivity indicates whether we have
seen CD8+ T cell reactivity in the three patients stained
so far (y = yes, TBD = to be determined).

| | | NetMHC | | | | % | Patient |
|---|---|---|---|---|---|---|---|
| | | 4.0 | | | EpiScan | Peptide | tetramer |
| Peptide | # | (nM) | MSi | ESP | MFI | Exchange | positivity? |
| SLPGVFCGV | 26 | 24.1 | 0.9797 | 0.99892 | 6.418 | 98.00 | y |
| NLIDSYFW | 27 | 5.9 | 0.1457 | 0.9335 | 5.971 | 98.40 | y |
| VMAYITGGVV | 29 | 482.2 | 0.1130 | N/A | 4.641 | 99.07 | y |
| TLIGDCATV | 31 | 17.6 | 0.6984 | 0.9962 | 3.866 | 98.16 | y |
| VLYQDVNCTEV | 23 | 546 | 0.9854 | N/A | 5.598 | 97.92 | TBD |
| VMVELVAEL | 24 | 12.3 | 0.9817 | 0.9970 | 5.338 | 98.63 | y |
| YIDIGNYTV | 25 | 10.6 | 0.7215 | 0.9077 | 4.421 | 97.94 | TBD |
| VMAYITGGV | 28 | 37.8 | 0.6562 | 0.9319 | 4.528 | 99.07 | TBD |
| AMDEFIERYKL | 30 | 290.6 | 0.9339 | N/A | 2.352 | 96.89 | TBD |
| TLATHGLAAV | 32 | 47.6 | 0.9669 | N/A | 4.040 | 97.98 | TBD |

#, SEQ ID NO:

### Example 5. Comparisons of EpiScan with and without HM13 Knockout

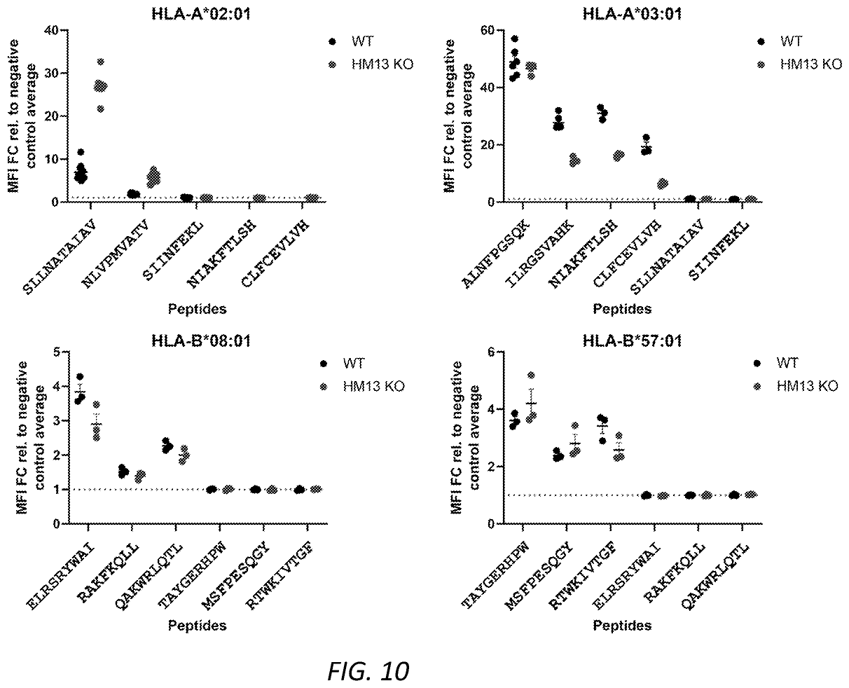

We evaluated the effect of knocking out the signal peptide peptidase HM13. As shown in FIG. 10A, the results indicated that HM13 knockout was only beneficial for HLA-A*02:01 signal:noise. The likely explanation for this is that HM13 activity in the ER generates short peptide fragments by cleaving signal peptides out of the ER membrane. Given the amino acid composition of signal peptides, these HM13-generated short peptides are only good substrates for HLA-A*02:01, and not the other alleles; thus, knockout lowers the background signal.

In addition, when the sequences of the HLA-A*02:01 ligands identified by WT EpiScan, HM13 KO EpiScan, and mass spectrometry, were compared, the results (FIG. 16) showed that HM13 knockout identified more L-ended peptides relative to WT, more similar to what is seen with mass spectrometry.

### Example 6. Comparison of Affinity of L- to V-Ended 9Mers Via EpiScan

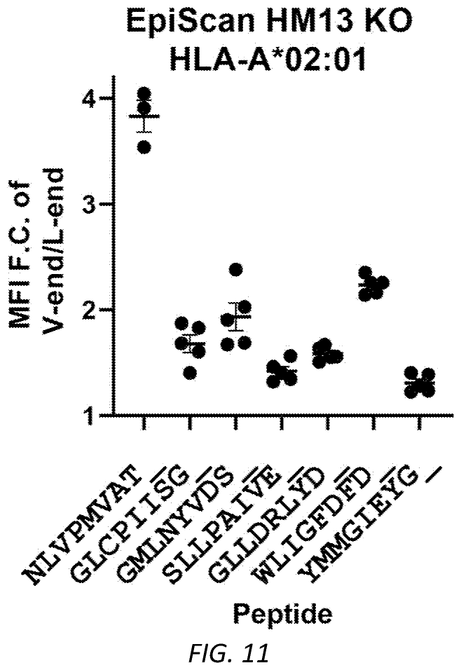

We compared the affinity of L- to V-ended 9mers via EpiScan. As shown in FIG. 11, the results indicated that V-ended 9mers are of higher affinity when binding to HLA-A*02:01 than L-ended 9mers. This would explain why more V-ended peptides were seen in WT EpiScan as opposed to HM13 EpiScan, and in comparison to mass spectrometry.

### Example 7. Confirmation of Signal Peptidase Cleavage Fidelity

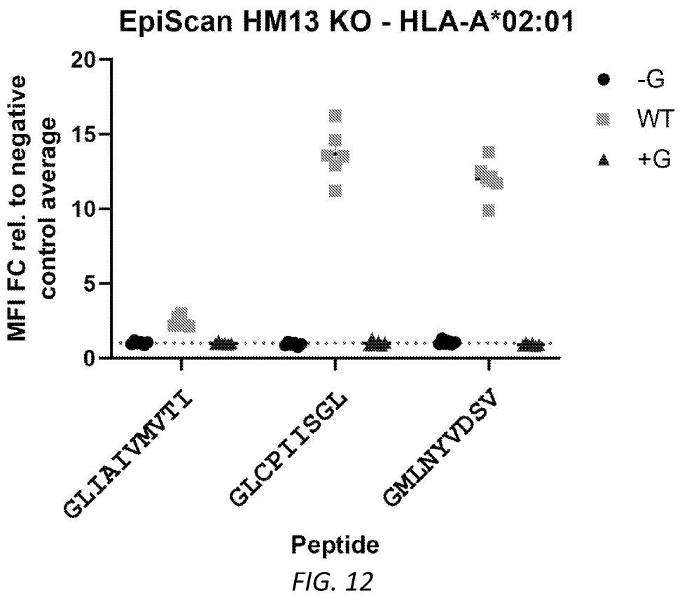

To confirm signal peptidase cleavage fidelity, we sought to challenge the system with peptides that would be most likely to be cleaved at the improper location. Thus, we chose three peptides known to bind HLA-A*02:01 that start with a glycine, which is also the last residue of the signal peptide, and included variants of each peptide with the initial glycine removed, or an additional glycine added. If the signal peptidase cleaves “too early”, leaving the last glycine of the signal peptide, then the removed glycine variant will cause an increase in surface MHC-I. Alternatively, if the signal peptides cleaves “too late”, removing an additional glycine, then the added glycine variant will cause an increase in surface MHC-I. If signal peptidase cleavage happens consistently, and precisely, at the end of the signal peptide then only the WT version of the peptides will lead to surface MHC-I signal. The results, shown in FIG. 12, indicated that the signal peptidase cleaves at precisely the desired location despite the signal peptide also ending in a glycine.

### Example 8. EpiScan Screens can be Performed by Magnetic-Activated Cell Sorting (MACS)

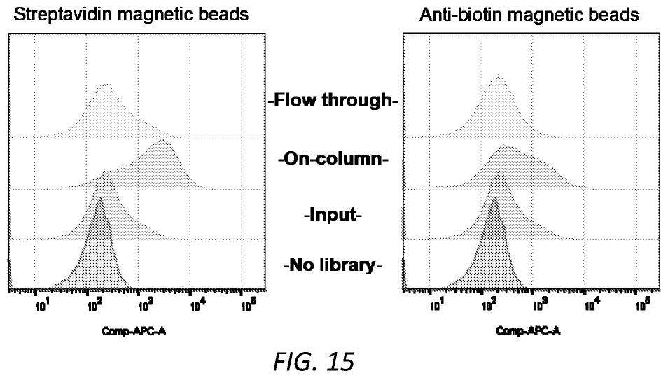

A diverse set of 200,000 distinct peptides was introduced into HLA-A*02:01 HM13 KO EpiScan cells. After selection, MACS was performed using a biotin-conjugated β2m antibody on 100 million cells for each condition, and the column flow through and the cells captured by the column were plated after sorting. For capture, both streptavidin (FIG. 15, left) and anti-biotin (FIG. 15, right) were used. Two days later the cells were stained with APC-anti-HLA-A*02:01 antibody and an increase in cell surface MHC-I was measured by flow cytometry. We saw a significant increase in surface MHC-I for the cells captured on-column, compared to both input and flow through, with either streptavidin or anti-biotin magnetic beads. Thus, MACS can be used, independent of FACS, to identify peptide:MHC-I complexes.

MACS allows more cells to be sorted in a shorter period of time than FACS. Thus, the success of MACS at isolating EpiScan cells that express higher affinity peptides permits larger scale screening of EpiScan peptide libraries.

### Example 9. Mass Spectrometry for MHC-I Peptides Via Conventional ORF Transfection Versus EpiScan

We wanted to determine whether mass spectrometry (MS) could be used in tandem with EpiScan for more efficient MS-based determination of MHC-I ligands from a particular pathogen or other set of potential antigens. For comparison, we also sought to compare to a more conventional “targeted” MS approach wherein ORFs from the pathogen of interest are transfected into a cell line containing just one HLA-I allele. Thus, we transfected 293T cells engineered to only express HLA-A*02:01 with SARS-CoV-2 ORFs corresponding to ORF1a/b, M, N, and S, then harvested the cells for MS two days later. In parallel, we performed an EpiScan screen with HLA-A*02:01 and a SARS-CoV-2 library with all possible 9-, 10-, and 11-mers. For this purpose, the EpiScan cells bearing the SARS-CoV-2 library were sorted in one bin based on surface MHC-I. After recovering from sorting, the cells were expanded and then harvested for MS.

We found that conducting MS on the EpiScan sorted cells was much more efficient than ORF transfection at identifying potential SARS-CoV-2 epitopes. MS of eluted MHC-I ligands discovered 214 high-confidence SARS-CoV-2 peptides out of a total of 457 peptides for the EpiScan cells. However, for the ORF transfected cells, MS of eluted MHC-I ligands discovered 1 high-confidence SARS-CoV-2 peptide out of a total of 3130 peptides. Thus, MS, in combination with EpiScan, can be used to identify MHC-I ligands in a high-throughput fashion.

| | | EpiScan SARS-CoV-2 screen |
|---|---|---|
| | ORF transfection (293T | (EpiScan cell with only |
| | with only HLA-A*02:01) | HLA-A*02:01) |
| SARS-CoV-2 | 1 | 214 |
| peptides | | |
| Total peptides | 3130 | 457 |

### Example 10. EpiScan can be Used to Directly Elicit CD8 T-Cell Responses

An assay for discovery of CD8 T cell epitopes known as T-Scan has been described (Kula et al., Cell. 2019 Aug. 8; 178(4):1016-1028.e13). When a T cell recognizes its cognate antigen on MHC-I, it releases granzyme to lyse the target cell. T-Scan relies on a Granzyme B (GzB) reporter that is activated after a CD8 T cell recognizes it. Here, T-Scan reporter cells have been engineered via TAP1/2 KO and HM13 KO to also be EpiScan cells. These EpiScan cells with the T-Scan reporter are referred to as EpiTScan cells. With EpiTScan we can precisely identify the specific peptide epitope responsible for T cell activation. Previously, T-Scan cells expressed short ORFs that were subject to endogenous processing and presentation and the short peptides responsible for T cell responses were inferred via prediction algorithms.

For this experiment, primary T cells were infected with a virus comprising a sequence for a human T cell Receptor (TCR), NLV3, that is specific to the peptide NLVPMVATV (SEQ ID NO:34), then those T cells were incubated together for 16 h at a 1:1 ratio with EpiTScan cells that express NLVPMVATV (SEQ ID NO:34) (FIG. 16, Epi pp65, far left) via the EpiScan Vector, or two negative control peptides via the EpiScan Vector (FIG. 16, Epi SAV10 and SIIN), no peptide at all (FIG. 16, neg), or NLVPMVATV (SEQ ID NO:34) was added directly to the media (FIG. 16, pulsed pp65). In the top graph of FIG. 16, the Granzyme reporter in the EpiTScan cells was measured. As expected, both pulsed peptide and EpiScan Vector expressed pp65 cause the NLV3 T cells to activate the GzB reporter. The bottom two graphs of FIG. 16 are different measures of T cell activation. The middle of FIG. 16, trogocytosis, was measured by the transfer of BFP from the cytoplasm of EpiTScan cells to the T-cells; BFP transfer indicated successful synapse formation between the T cell and the EpiTScan cells. CD69 (bottom) is a T cell activation marker. Here, CD69 surface staining on the T cells was highest in the pp65 conditions. Background CD69 staining was expected based on how the T cells were stimulated prior to infection with the NLV3 TCR.

These results show that the EpiScan cells are capable of eliciting an immune response, as demonstrated by previously published metrics (TScan GzB reporter, Trogocytosis, and CD69).

### REFERENCES

1. Chaplin, D. D. Overview of the immune response. J. Allergy Clin. Immunol. 125, S3-23 (2010).
2. Gfeller, D. & Bassani-Sternberg, M. Predicting antigen presentation-What could we learn from a million peptides? Frontiers in Immunology 9, 1716 (2018).
3. Walz, S. et al. The antigenic landscape of multiple myeloma: Mass spectrometry (re)defines targets for T-cell-based immunotherapy. Blood 126, 1203-1213 (2015).
4. Sarkizova, S. et al. A large peptidome dataset improves HLA class I epitope prediction across most of the human population. Nat. Biotechnol. 38, 199-209 (2020).
5. Momburg, F. & Hammerling, G. J. Generation and TAP-Mediated Transport of Peptides for Major Histocompatibility Complex Class I Molecules. Adv.

Immunol. 68, 191-256 (1998).
6. Serwold, T., Gonzalez, F., Kim, J., Jacob, R. & Shastri, N. ERAAP customizes peptides for MHC class I molecules in the endoplasmic reticulum. Nature 419, 480-483 (2002).
7. Saveanu, L. et al. Concerted peptide trimming by human ERAP1 and ERAP2 aminopeptidase complexes in the endoplasmic reticulum. Nat. Immunol. 6, 689-697 (2005).
8. Gejman, R. S. et al. Rejection of immunogenic tumor clones is limited by clonal fraction. Elife 7, 1-22 (2018).
9. Porgador, A., Yewdell, J. W., Deng, Y., Bennink, J. R. & Germain, R. N. Localization, quantitation, and in situ detection of specific peptide-MHC class I complexes using a monoclonal antibody. Immunity 6, 715-26 (1997).
10. Nijman, H. W. et al. Identification of peptide sequences that potentially trigger HLA-A2.1-restricted cytotoxic T lymphocytes. Eur. J. Immunol. 23, 1215-1219 (1993).
11. Vita, R. et al. The immune epitope database (IEDB) 3.0. Nucleic Acids Res. 43, D405-D412 (2015).
12. Bassani-Sternberg, M., Pletscher-Frankild, S., Jensen, L. J. & Mann, M. Mass spectrometry of human leukocyte antigen class i peptidomes reveals strong effects of protein abundance and turnover on antigen presentation. Mol. Cell. Proteomics 14, 658-673 (2015).
13. Yuen, G. J., Weller, S. & Pakes, G. E. A Review of the Pharmacokinetics of Abacavir. Clin. Pharmacokinet. 47, 351-371 (2008).
14. Martin, A. M. et al. Predisposition to abacavir hypersensitivity conferred by HLA-B*5701 and a haplotypic Hsp70-Hom variant. Proc. Natl. Acad. Sci. U.S.A 101, 4180-5 (2004).
15. Ostrov, D. A. et al. Drug hypersensitivity caused by alteration of the MHC-presented self-peptide repertoire. Proc. Natl. Acad. Sci. U.S.A 109, 9959-64 (2012).
16. Harris, J. L., Alper, P. B., Li, J., Rechsteiner, M. & Backes, B. J. Substrate specificity of the human proteasome. Chem. Biol. 8, 1131-1141 (2001).
17. Abelin, J. G. et al. Mass Spectrometry Profiling of HLA-Associated Peptidomes in Mono-allelic Cells Enables More Accurate Epitope Prediction. Immunity 46, 315-326 (2017).
18. Andreatta, M. & Nielsen, M. Gapped sequence alignment using artificial neural networks: application to the MHC class I system. Bioinformatics 32, 511-517 (2016).
19. Nielsen, M. et al. Reliable prediction of T-cell epitopes using neural networks with novel sequence representations—Nielsen—2009—Protein Science—Wiley Online Library. Protein Sci. 12, 1007-1017 (2003).
20. Andreatta, M. & Nielsen, M. Gapped sequence alignment using artificial neural networks: Application to the MHC class i system. Bioinformatics 32, 511-517 (2015).
21. O'Donnell, T. J. et al. MHCflurry: Open-Source Class I MHC Binding Affinity Prediction. Cell Syst. 7, 129-132.e4 (2018).
22. Zhang, X. et al. Viral and host factors related to the clinical outcome of COVID-19. Nature 1-7 (2020).
23. Meckiff, B. J. et al. Single-cell transcriptomic analysis of SARS-CoV-2 reactive CD4+ T cells. bioRxiv 2020.06.12.148916 (2020).
24. Takahashi, T. et al. Sex differences in immune responses to SARS-CoV-2 that underlie disease outcomes. medRxiv 2020.06.06.20123414 (2020).
25. Toussaint, N. C., Maman, Y., Kohlbacher, O. & Louzoun, Y. Universal peptide vaccines—Optimal peptide vaccine design based on viral sequence conservation. Vaccine 29, 8745-8753 (2011).
26. Ashkenazy, H. et al. ConSurf 2016: an improved methodology to estimate and visualize evolutionary conservation in macromolecules. Nucleic Acids Res. 44, W344-W350 (2016).
27. Celniker, G. et al. ConSurf: Using evolutionary data to raise testable hypotheses about protein function. Israel Journal of Chemistry 53, 199-206 (2013).
28. Le Bert, N. et al. SARS-CoV-2-specific T cell immunity in cases of COVID-19 and SARS, and uninfected controls. Nature 2020.05.26.115832 (2020).
29. Croft, N. P. et al. Most viral peptides displayed by class I MHC on infected cells are immunogenic. Proc. Natl. Acad. Sci. U.S.A 116, 3112-3117 (2019).
30. Scott, D. W. & De Groot, A. S. Can we prevent immunogenicity of human protein drugs? Annals of the Rheumatic Diseases 69, (2010).
31. Yewdell, J. W. Confronting Complexity: Real-World Immunodominance in Antiviral CD8+ T Cell Responses. Immunity 25, 533-543 (2006).
32. Panagioti, E., Klenerman, P., Lee, L. N., van der Burg, S. H. & Arens, R. Features of effective T cell-inducing vaccines against chronic viral infections. Frontiers in Immunology 9, 276 (2018).
33. Hu, Z., Ott, P. A. & Wu, C. J. Towards personalized, tumour-specific, therapeutic vaccines for cancer. Nat. Rev. Immunol. 18, 168-182 (2018).
34. Kim, Y., Sidney, J., Pinilla, C., Sette, A. & Peters, B. Derivation of an amino acid similarity matrix for peptide:MHC binding and its application as a Bayesian prior. BMC Bioinformatics 10, 394 (2009).
35. Bremel, R. D. & Homan, E. J. An integrated approach to epitope analysis I: Dimensional reduction, visualization and prediction of MHC binding using amino acid principal components and regression approaches. Immunome Res. 6, 7 (2010).
36. Ashkenazy, H., Erez, E., Martz, E., Pupko, T. & Ben-Tal, N. ConSurf 2010: Calculating evolutionary conservation in sequence and structure of proteins and nucleic acids. Nucleic Acids Res. 38, (2010).
37. Thomsen, M. C. F. & Nielsen, M. Seq2Logo: a method for construction and visualization of amino acid binding motifs and sequence profiles including sequence weighting, pseudo counts and two-sided representation of amino acid enrichment and depletion. Nucleic Acids Res. 40, W281-W287 (2012).
38. M. C. Verweij, et al., The Capacity of UL49.5 Proteins To Inhibit TAP Is Widely Distributed among Members of the Genus Varicellovirus. J. Virol. 85, 2351-2363 (2011).
39. M. C. Verweij, et al., Viral Inhibition of the Transporter Associated with Antigen Processing (TAP): A Striking Example of Functional Convergent Evolution. PLoS Pathog. 11, 1-19 (2015).
40. D. Koppers-Lalic, et al., Varicelloviruses avoid T cell recognition by UL49.5-mediated inactivation of the transporter associated with antigen processing. Proc. Natl. Acad. Sci. 102, 5144-5149 (2005).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated cell, wherein the cell has been engineered or modified to lack expression of two, three, four, or more, preferably all, of human leukocyte antigen A (HLA-A); HLA-B; HLA-C; Transporter 1, ATP Binding Cassette Subfamily B Member 1 (TAP1); TAP2; endoplasmic reticulum aminopeptidase 1 (ERAP1); ERAP2; and histocompatibility minor 13 (HM13), and wherein the cell expresses a single HLA allele.

2. The isolated cell of claim 1, which lacks expression of TAP1; TAP2; ERAP1; ERAP2; and HM13; and lacks expression of at least two of HLA-A; HLA-B; and HLA-C.

3. The isolated cell of claim 1, which lacks expression of TAP1; TAP2; ERAP1; ERAP2; HM13; HLA-A; HLA-B; HLA-C, and expresses an exogenous HLA-I allele.

4. The isolated cell of claim 1, which is a human cell.

5. The isolated cell of claim 1, further comprising (i) a nucleic acid comprising one or more sequences encoding candidate epitope peptides linked to a signal peptide that directs the peptide to the endoplasmic reticulum (ER), and a promoter that drives expression of the candidate epitope peptide linked to a signal peptide; or (ii) candidate epitope peptides linked to a signal peptide that directs the peptide to the ER.

6. The isolated cell of claim 1, wherein the signal peptide comprises a MMTV gp70 signal peptide.

7. The isolated cell of claim 5, wherein the cell expresses the candidate epitope peptides linked to a signal peptide, and the candidate epitope peptides are trafficked to the ER.

8. A method for identifying an MHC-I binding peptide, the method comprising:
providing a sample comprising the cells of claim 1 that express a selected MHC-I allele;
expressing in the cells a plurality of different candidate epitope peptides, such that each cell expresses a single selected candidate epitope peptide or plurality of candidate epitope peptides;
isolating cells that have cell surface expression of the MHC-I allele; and
identifying candidate epitope peptides in the cells that have cell surface expression of the MHC-I allele, thereby identifying peptides that bind to the MHC-I allele.

9. The method of claim 8, wherein expressing in the cells a plurality of different candidate epitope peptides comprises contacting the cells with a plurality of nucleic acids each comprising one or more sequences encoding candidate epitope peptides linked to a signal peptide that directs the peptide to the endoplasmic reticulum (ER), and a promoter that drives expression of the candidate epitope peptide linked to the signal peptide, under conditions sufficient for the cells to express the peptides, preferably wherein the signal peptide comprises a MMTV gp70 signal peptide.

10. The method of claim 9, wherein the nucleic acids comprise expression vectors.

11. The method of claim 10, wherein the expression vectors are viral expression vectors or plasmids.

12. The method of claim 11, wherein the viral expression vectors are retroviral, preferably lentiviral, vectors.

13. The method of claim 8, wherein each cell expresses one to 100 or more different candidate epitope peptides.

14. The method of claim 8, wherein the plurality of different candidate epitope peptides comprise random sequences.

15. The method of claim 8, wherein the plurality of different candidate epitope peptides comprise sequences derived from a pathogen, preferably a viral, bacterial, parasitic, or fungal pathogen, or from a cancer antigen.

16. The method of claim 8, wherein the plurality of different candidate epitope peptides comprise a peptidome for an organism, or sequences from an autoantigen or potential autoantigen.

17. The method of claim 14, wherein the plurality of different candidate epitope peptides comprises at least 100 or more different candidate epitope peptides.

18. The method of claim 8, wherein isolating cells that have cell surface expression of an MHC allele comprises using fluorescence-activated cell sorting (FACS) or magnetic-activated cell sorting (MACS).

19. The method of claim 8, wherein identifying candidate epitope peptides comprises determining sequences encoding the peptides expressed in the cells that have cell surface expression of an MHC allele.

20. The method of claim 19, wherein the sequences encoding the peptides are determined by sequencing.

21. A method of isolating a cell for use in generating an immune response to an epitope in a subject, the method comprising providing a sample comprising the cells of claim 1 that express a selected MHC-I allele;
expressing in the cells a plurality of different candidate epitope peptides linked to a signal peptide that directs the peptide to the endoplasmic reticulum (ER), such that each cell expresses a single selected candidate epitope peptide or plurality of candidate epitope peptides; and
isolating cells that have cell surface expression of the MHC-I allele.

22. The method of claim 21, wherein the plurality of different candidate epitope peptides comprise sequences derived from a pathogen, preferably a viral, bacterial, parasitic, or fungal pathogen, or from a cancer antigen.

23. A method for stimulating T cells, the method comprising:
providing a sample comprising the cells of claim 1 that express a selected MHC-I allele;
expressing in the cells one or more specific epitope peptides linked to a signal peptide that directs the peptide to the endoplasmic reticulum (ER), such that each cell expresses a single specific epitope peptide or plurality of specific epitope peptides;
incubating the cells in the presence of T cells in culture under conditions that allow activation of the T cells; and
isolating activated T cells from the culture.

24. The method of claim 23, wherein the specific epitope peptides comprise sequences derived from a pathogen, preferably a viral, bacterial, parasitic, or fungal pathogen, or from a cancer antigen.


SEQUENCE LISTING (234 sequences)

SEQ ID NO: 1 - DNA, 20 bp, Unknown
  Description of Unknown: target sequence
gccatgcgagagaagctccg

SEQ ID NO: 2 - DNA, 20 bp, Unknown
  Description of Unknown: target sequence
agttcgaagctttgccaacg

SEQ ID NO: 3 - DNA, 20 bp, Unknown
  Description of Unknown: target sequence
atccccatatatgtatacca

SEQ ID NO: 4 - DNA, 20 bp, Unknown
  Description of Unknown: target sequence
acaacaaagtcttgatgtgg

SEQ ID NO: 5 - DNA, 20 bp, Unknown
  Description of Unknown: target sequence
cggctactacaaccagagcg

SEQ ID NO: 6 - DNA, 21 bp, Unknown
  Description of Unknown: target sequence
gagatcacactgacctggcag

SEQ ID NO: 7 - DNA, 20 bp, Unknown
  Description of Unknown: target sequence
agattatgcactggatgctg

SEQ ID NO: 8 - DNA, 20 bp, Unknown
  Description of Unknown: target sequence
gtgcaatttgctcctgacgg

SEQ ID NO: 9 - DNA, 20 bp, Unknown
  Description of Unknown: target sequence
aaggccattctagctgcagt

SEQ ID NO: 10 - DNA, 21 bp, Unknown
  Description of Unknown: target sequence
gagatgcaacaaagtccagag

SEQ ID NO: 11 - DNA, 20 bp, Unknown
  Description of Unknown: target sequence
gcctcacctgaaatactatg

SEQ ID NO: 12 - DNA, 20 bp, Unknown
  Description of Unknown: target sequence
gccccaccaacagcactacg

SEQ ID NO: 13 - DNA, 20 bp, Unknown
  Description of Unknown: target sequence
agaaatacatggacagcagg

SEQ ID NO: 14 - DNA, 20 bp, Unknown
  Description of Unknown: target sequence
ggtatttggcaccaatgtga

SEQ ID NO: 15 - protein, 98 aa, Artificial Sequence
  Description of Artificial Sequence: Synthetic polypeptide
MPNHQSGSPTGSSDLLLDGKKQRAHLALRRKRRREMRKINRKVRRMNLAPIKEKTAWQHLQALIFEAEEVLKTSQTPQTSLTLFLALLAVLAPPPVSG

SEQ ID NO: 16 - DNA, 57 bp, Artificial Sequence
  Description of Artificial Sequence: Synthetic oligonucleotide
  modified_base (16)..(17): a, c, t, g, unknown or other
  modified_base (19)..(20): a, c, t, g, unknown or other
  modified_base (22)..(23): a, c, t, g, unknown or other
  modified_base (25)..(26): a, c, t, g, unknown or other
  modified_base (28)..(29): a, c, t, g, unknown or other
  modified_base (31)..(32): a, c, t, g, unknown or other
  modified_base (34)..(35): a, c, t, g, unknown or other
  modified_base (37)..(38): a, c, t, g, unknown or other
  modified_base (40)..(41): a, c, t, g, unknown or other
ccacctgtgagcgggnnbnnbnnbnnbnnbnnbnnbnnbnnbtaagcacgttactgg

SEQ ID NO: 17 - DNA, 32 bp, Artificial Sequence
  Description of Artificial Sequence: Synthetic primer
tggccgtattggccccgccacctgtgagcggg

SEQ ID NO: 18 - DNA, 32 bp, Artificial Sequence
  Description of Artificial Sequence: Synthetic primer
attccaagcggcttcggccagtaacgtgctta

SEQ ID NO: 19 - DNA, 49 bp, Artificial Sequence
  Description of Artificial Sequence: Synthetic oligonucleotide
tccctacacgacgctcttccgatcttacagctcgccacctgtgagcggg

SEQ ID NO: 20 - DNA, 20 bp, Artificial Sequence
  Description of Artificial Sequence: Synthetic oligonucleotide
ggcttcggccagtaacgtgc

SEQ ID NO: 21 - DNA, 54 bp, Artificial Sequence
  Description of Artificial Sequence: Synthetic oligonucleotide
aatgatacggcgaccaccgagatctacactctttccctacacgacgctcttccg

SEQ ID NO: 22 - DNA, 52 bp, Artificial Sequence
  Description of Artificial Sequence: Synthetic oligonucleotide
  modified_base (25)..(31): a, c, t, g, unknown or other
caagcagaagacggcatacgagatnnnnnnngtgactggagttcagacgtgt

SEQ ID NO: 23 - protein, 11 aa, Severe acute respiratory syndrome coronavirus 2
VLYQDVNCTEV

SEQ ID NO: 24 - protein, 9 aa, Severe acute respiratory syndrome coronavirus 2
VMVELVAEL

SEQ ID NO: 25 - protein, 9 aa, Severe acute respiratory syndrome coronavirus 2
YIDIGNYTV

SEQ ID NO: 26 - protein, 9 aa, Severe acute respiratory syndrome coronavirus 2
SLPGVFCGV

SEQ ID NO: 27 - protein, 9 aa, Severe acute respiratory syndrome coronavirus 2
NLIDSYFVV

SEQ ID NO: 28 - protein, 9 aa, Severe acute respiratory syndrome coronavirus 2
VMAYITGGV

SEQ ID NO: 29 - protein, 10 aa, Severe acute respiratory syndrome coronavirus 2
VMAYITGGVV

SEQ ID NO: 30 - protein, 11 aa, Severe acute respiratory syndrome coronavirus 2
AMDEFIERYKL

SEQ ID NO: 31 - protein, 9 aa, Severe acute respiratory syndrome coronavirus 2
TLIGDCATV

SEQ ID NO: 32 - protein, 10 aa, Severe acute respiratory syndrome coronavirus 2
TLATHGLAAV

SEQ ID NO: 33 - protein, 8 aa, Unknown
  Description of Unknown: short peptide sequence
SIINFEKL

SEQ ID NO: 34 - protein, 9 aa, Unknown
  Description of Unknown: short peptide sequence
NLVPMVATV

SEQ ID NO: 35 - protein, 9 aa, Unknown
  Description of Unknown: short peptide sequence
CLFCEVLVH

SEQ ID NO: 36 - protein, 9 aa, Unknown
  Description of Unknown: short peptide sequence
RCFQWALMY

SEQ ID NO: 37 - protein, 9 aa, Unknown
  Description of Unknown: short peptide sequence
LTCSLLLWH

SEQ ID NO: 38 - protein, 9 aa, Unknown
  Description of Unknown: short peptide sequence
RLCSDVWLH

SEQ ID NO: 39 - protein, 9 aa, Unknown
  Description of Unknown: short peptide sequence
MTCARVLCH

SEQ ID NO: 40 - protein, 9 aa, Unknown
  Description of Unknown: short peptide sequence
TVSSIILRH

SEQ ID NO: 41 - protein, 9 aa, Unknown
  Description of Unknown: short peptide sequence
NIAKFTLSH

SEQ ID NO: 42 - protein, 9 aa, Severe acute respiratory syndrome coronavirus 2
ATSRTLSYY

SEQ ID NO: 43 - protein, 10 aa, Severe acute respiratory syndrome coronavirus 2
KFPRGQGVPI

SEQ ID NO: 44 - protein, 9 aa, Severe acute respiratory syndrome coronavirus 2
NPANNAAIV

SEQ ID NO: 45 - protein, 9 aa, Severe acute respiratory syndrome coronavirus 2
VPHVGEIPV

SEQ ID NO: 46 - protein, 9 aa, Severe acute respiratory syndrome coronavirus 2
YPLECIKDL

SEQ ID NO: 47 - protein, 10 aa, Severe acute respiratory syndrome coronavirus 2
YPQVNGLTSI

SEQ ID NO: 48 - protein, 9 aa, Severe acute respiratory syndrome coronavirus 2
LACEDLKPV

SEQ ID NO: 49 - protein, 9 aa, Severe acute respiratory syndrome coronavirus 2
VPMEKLKTL

SEQ ID NO: 50 - protein, 9 aa, Severe acute respiratory syndrome coronavirus 2
VAKSHSIAL

SEQ ID NO: 51 - protein, 9 aa, Severe acute respiratory syndrome coronavirus 2
MPASWVMRI

SEQ ID NO: 52 - protein, 11 aa, Severe acute respiratory syndrome coronavirus 2
KMADQAMTQMY

SEQ ID NO: 53 - protein, 9 aa, Severe acute respiratory syndrome coronavirus 2
CTDDNALAY

SEQ ID NO: 54 - protein, 9 aa, Severe acute respiratory syndrome coronavirus 2
VTANVNALL

SEQ ID NO: 55 - protein, 10 aa, Severe acute respiratory syndrome coronavirus 2
LAIDAYPLTK

SEQ ID NO: 56 - protein, 9 aa, Severe acute respiratory syndrome coronavirus 2
AIDAYPLTK

SEQ ID NO: 57 - protein, 9 aa, Severe acute respiratory syndrome coronavirus 2
TPHTVLQAV

SEQ ID NO: 58 - protein, 9 aa, Severe acute respiratory syndrome coronavirus 2
ALCEKALKY

SEQ ID NO: 59 - protein, 9 aa, Severe acute respiratory syndrome coronavirus 2
LPIDKCSRI

SEQ ID NO: 60 - protein, 10 aa, Severe acute respiratory syndrome coronavirus 2
KSAQCFKMFY

SEQ ID NO: 61 - protein, 9 aa, Severe acute respiratory syndrome coronavirus 2
SPYNSQNAV

SEQ ID NO: 62 - protein, 10 aa, Severe acute respiratory syndrome coronavirus 2
TVDSSQGSEY

SEQ ID NO: 63 - protein, 9 aa, Severe acute respiratory syndrome coronavirus 2
IPLMYKGLL

SEQ ID NO: 64 - protein, 11 aa, Severe acute respiratory syndrome coronavirus 2
TYACWHHSIGF

SEQ ID NO: 65 - protein, 10 aa, Severe acute respiratory syndrome coronavirus 2
DAIMTRCLAV

SEQ ID NO: 66 - protein, 9 aa, Severe acute respiratory syndrome coronavirus 2
KRVDWTIEY

SEQ ID NO: 67 - protein, 9 aa, Severe acute respiratory syndrome coronavirus 2
VPLKSATCI

SEQ ID NO: 68 - protein, 9 aa, Severe acute respiratory syndrome coronavirus 2
IMRTFKVSI

SEQ ID NO: 69 - protein, 9 aa, Severe acute respiratory syndrome coronavirus 2
IIKNLSKSL

SEQ ID NO: 70 - protein, 10 aa, Severe acute respiratory syndrome coronavirus 2
IPYNSVTSSI

SEQ ID NO: 71 - protein, 11 aa, Severe acute respiratory syndrome coronavirus 2
IPYNSVTSSIV

SEQ ID NO: 72 - protein, 9 aa, Severe acute respiratory syndrome coronavirus 2
IVNNATNVV

SEQ ID NO: 73 - protein, 9 aa, Severe acute respiratory syndrome coronavirus 2
SANNCTFEY

SEQ ID NO: 74 - protein, 9 aa, Severe acute respiratory syndrome coronavirus 2
IPTNFTISV

SEQ ID NO: 75 - protein, 9 aa, Severe acute respiratory syndrome coronavirus 2
VYDPLQPEL

SEQ ID NO: 76 - protein, 9 aa, Severe acute respiratory syndrome coronavirus 2
TLATCELYH

SEQ ID NO: 77 - protein, 9 aa, Severe acute respiratory syndrome coronavirus 2
SWFTALTQH

SEQ ID NO: 78 - protein, 10 aa, Severe acute respiratory syndrome coronavirus 2
TMCDIRQLLF

SEQ ID NO: 79 - protein, 9 aa, Severe acute respiratory syndrome coronavirus 2
VYIGDPAQL

SEQ ID NO: 80 - protein, 9 aa, Severe acute respiratory syndrome coronavirus 2
YYSLLMPIL

SEQ ID NO: 81 - protein, 9 aa, Severe acute respiratory syndrome coronavirus 2
KYTQLCQYL

SEQ ID NO: 82 - protein, 9 aa, Severe acute respiratory syndrome coronavirus 2
VFVLWAHGF

SEQ ID NO: 83 - protein, 11 aa, Severe acute respiratory syndrome coronavirus 2
YYSLLMPILTL

SEQ ID NO: 84 - protein, 9 aa, Severe acute respiratory syndrome coronavirus 2
YFIKGLNNL

SEQ ID NO: 85 - protein, 10 aa, Severe acute respiratory syndrome coronavirus 2
TVDSSQGSEY

SEQ ID NO: 86 - protein, 9 aa, Severe acute respiratory syndrome coronavirus 2
AIDAYPLTK

SEQ ID NO: 87 - protein, 10 aa, Severe acute respiratory syndrome coronavirus 2
IVDTVSALVY

SEQ ID NO: 88 - protein, 10 aa, Severe acute respiratory syndrome coronavirus 2
MADQAMTQMY

SEQ ID NO: 89 - protein, 9 aa, Severe acute respiratory syndrome coronavirus 2
VTDVTQLYL

SEQ ID NO: 90 - protein, 10 aa, Severe acute respiratory syndrome coronavirus 2
ATEETFKLSY

SEQ ID NO: 91 - protein, 10 aa, Severe acute respiratory syndrome coronavirus 2
LAIDAYPLTK

SEQ ID NO: 92 - protein, 11 aa, Severe acute respiratory syndrome coronavirus 2
ESFGGASCCLY

SEQ ID NO: 93 - protein, 11 aa, Severe acute respiratory syndrome coronavirus 2
AIDAYPLTKHP

SEQ ID NO: 94 - protein, 11 aa, Severe acute respiratory syndrome coronavirus 2
KATEETFKLSY

SEQ ID NO: 95 - protein, 11 aa, Severe acute respiratory syndrome coronavirus 2
KMADQAMTQMY

SEQ ID NO: 96 - protein, 11 aa, Severe acute respiratory syndrome coronavirus 2
SMMILSDDAVV

SEQ ID NO: 97 - protein, 9 aa, Severe acute respiratory syndrome coronavirus 2
YLNTLTLAV

SEQ ID NO: 98 - protein, 11 aa, Severe acute respiratory syndrome coronavirus 2
TMCDIRQLLFV

SEQ ID NO: 99 - protein, 9 aa, Severe acute respiratory syndrome coronavirus 2
TMADLVYAL

SEQ ID NO: 100 - protein, 9 aa, Severe acute respiratory syndrome coronavirus 2
RLANECAQV

SEQ ID NO: 101 - protein, 11 aa, Severe acute respiratory syndrome coronavirus 2
VQQWGFTGNLQ

SEQ ID NO: 102 - protein, 9 aa, Severe acute respiratory syndrome coronavirus 2
ELPTGVHAG

SEQ ID NO: 103 - protein, 10 aa, Severe acute respiratory syndrome coronavirus 2
KCTSVVLLSV

SEQ ID NO: 104 - protein, 9 aa, Severe acute respiratory syndrome coronavirus 2
IMASLVLAR

SEQ ID NO: 105 - protein, 10 aa, Severe acute respiratory syndrome coronavirus 2
IMASLVLARK

SEQ ID NO: 106 - protein, 11 aa, Severe acute respiratory syndrome coronavirus 2
RIMASLVLARK

SEQ ID NO: 107 - protein, 9 aa, Severe acute respiratory syndrome coronavirus 2
AIDAYPLTK

SEQ ID NO: 108 - protein, 10 aa, Severe acute respiratory syndrome coronavirus 2
QTMLFTMLRK

SEQ ID NO: 109 - protein, 9 aa, Severe acute respiratory syndrome coronavirus 2
TMLFTMLRK

SEQ ID NO: 110 - protein, 9 aa, Severe acute respiratory syndrome coronavirus 2
VLHDIGNPK

SEQ ID NO: 111 - protein, 11 aa, Severe acute respiratory syndrome coronavirus 2
MADQAMTQMYK

SEQ ID NO: 112 - protein, 9 aa, Severe acute respiratory syndrome coronavirus 2
SICSTMTNR

SEQ ID NO: 113 - protein, 10 aa, Severe acute respiratory syndrome coronavirus 2
LAIDAYPLTK

SEQ ID NO: 114 - protein, 11 aa, Severe acute respiratory syndrome coronavirus 2
KMADQAMTQMY

SEQ ID NO: 115 - protein, 9 aa, Severe acute respiratory syndrome coronavirus 2
MASLVLARK

SEQ ID NO: 116 - protein, 9 aa, Severe acute respiratory syndrome coronavirus 2
MTNRQFHQK

SEQ ID NO: 117 - protein, 9 aa, Severe acute respiratory syndrome coronavirus 2
RQFHQKLLK

SEQ ID NO: 118 - protein, 9 aa, Severe acute respiratory syndrome coronavirus 2
ATVVIGTSK

SEQ ID NO: 119 - protein, 10 aa, Severe acute respiratory syndrome coronavirus 2
DAIMTRCLAV

SEQ ID NO: 120 - protein, 11 aa, Severe acute respiratory syndrome coronavirus 2
MPNMLRIMASL

SEQ ID NO: 121 - protein, 11 aa, Severe acute respiratory syndrome coronavirus 2
APRTLLTKGTL

SEQ ID NO: 122 - protein, 9 aa, Severe acute respiratory syndrome coronavirus 2
MPNMLRIMA

SEQ ID NO: 123 - protein, 9 aa, Severe acute respiratory syndrome coronavirus 2
IPLMYKGLL

SEQ ID NO: 124 - protein, 9 aa, Severe acute respiratory syndrome coronavirus 2
SPYNSQNAV

SEQ ID NO: 125 - protein, 9 aa, Severe acute respiratory syndrome coronavirus 2
LPVNVAFEL

SEQ ID NO: 126 - protein, 9 aa, Severe acute respiratory syndrome coronavirus 2
SARIVYTAC

SEQ ID NO: 127 - protein, 9 aa, Severe acute respiratory syndrome coronavirus 2
ICQAVTANV

SEQ ID NO: 128 - protein, 9 aa, Severe acute respiratory syndrome coronavirus 2
VCRFDTRVL

SEQ ID NO: 129 - protein, 9 aa, Severe acute respiratory syndrome coronavirus 2
ITRAKVGIL

SEQ ID NO: 130 - protein, 9 aa, Severe acute respiratory syndrome coronavirus 2
LMIERFVSL

SEQ ID NO: 131 - protein, 11 aa, Severe acute respiratory syndrome coronavirus 2
YLRKHFSMMIL

SEQ ID NO: 132 - protein, 9 aa, Severe acute respiratory syndrome coronavirus 2
YLRKHFSMM

SEQ ID NO: 133 - protein, 10 aa, Severe acute respiratory syndrome coronavirus 2
DAIMTRCLAV

SEQ ID NO: 134 - protein, 9 aa, Severe acute respiratory syndrome coronavirus 2
TERLKLFAA

SEQ ID NO: 135 - protein, 10 aa, Severe acute respiratory syndrome coronavirus 2
TAYANSVFNI

SEQ ID NO: 136 - protein, 9 aa, Severe acute respiratory syndrome coronavirus 2
FPLCANGQV

SEQ ID NO: 137 - protein, 9 aa, Severe acute respiratory syndrome coronavirus 2
SPYNSQNAV

SEQ ID NO: 138 - protein, 9 aa, Severe acute respiratory syndrome coronavirus 2
VPYNMRVIH

SEQ ID NO: 139 - protein, 10 aa, Severe acute respiratory syndrome coronavirus 2
TVDSSQGSEY

SEQ ID NO: 140 - protein, 9 aa, Severe acute respiratory syndrome coronavirus 2
IPLMYKGLL

SEQ ID NO: 141 - protein, 9 aa, Severe acute respiratory syndrome coronavirus 2
WAHGFELTS

SEQ ID NO: 142 - protein, 11 aa, Severe acute respiratory syndrome coronavirus 2
VNVAFELWAKR

SEQ ID NO: 143 - protein, 11 aa, Severe acute respiratory syndrome coronavirus 2
VVFDEISMATN

SEQ ID NO: 144 - protein, 10 aa, Severe acute respiratory syndrome coronavirus 2
LIDLQELGKY

SEQ ID NO: 145 - protein, 9 aa, Severe acute respiratory syndrome coronavirus 2
AQALNTLVK

SEQ ID NO: 146 - protein, 11 aa, Severe acute respiratory syndrome coronavirus 2
RSFIEDLLFNK

SEQ ID NO: 147 - protein, 9 aa, Severe acute respiratory syndrome coronavirus 2
GIYQTSNFR

SEQ ID NO: 148 - protein, 9 aa, Severe acute respiratory syndrome coronavirus 2
AEIRASANL

SEQ ID NO: 149 - protein, 11 aa, Severe acute respiratory syndrome coronavirus 2
IEDLLFNKVTL

SEQ ID NO: 150 - protein, 9 aa, Severe acute respiratory syndrome coronavirus 2
IANQFNSAI

SEQ ID NO: 151 - protein, 10 aa, Severe acute respiratory syndrome coronavirus 2
MAYRFNGIGV

SEQ ID NO: 152 - protein, 10 aa, Severe acute respiratory syndrome coronavirus 2
RLQSLQTYVT

SEQ ID NO: 153 - protein, 9 aa, Unknown
  Description of Unknown: short peptide sequence
PRKLPKLGP

SEQ ID NO: 154 - protein, 11 aa, Unknown
  Description of Unknown: short peptide sequence
QLESIINFEKL

SEQ ID NO: 155 - protein, 13 aa, Unknown
  Description of Unknown: short peptide sequence
LEQLESIINFEKL

SEQ ID NO: 156 - protein, 5 aa, Unknown
  Description of Unknown: short peptide sequence
RDGCK

SEQ ID NO: 157 - protein, 10 aa, Unknown
  Description of Unknown: short peptide sequence
SLLNATAIAV

SEQ ID NO: 158 - protein, 10 aa, Unknown
  Description of Unknown: short peptide sequence
ANLVPMVATV

SEQ ID NO: 159 - protein, 16 aa, Unknown
  Description of Unknown: short peptide sequence
QAGILARNLVPMVATV

SEQ ID NO: 160 - protein, 9 aa, Unknown
  Description of Unknown: short peptide sequence
ALNFPGSQK

SEQ ID NO: 161-169 - skipped (9 entries)

SEQ ID NO: 170 - protein, 9 aa, Unknown
  Description of Unknown: short peptide sequence
ILRGSVAHK

SEQ ID NO: 171 - protein, 365 aa, Artificial Sequence
  Description of Artificial Sequence: Synthetic polypeptide
MAVMAPRTLVLLLSGALALTQTWAGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 172 - protein, 98 aa, Artificial Sequence; identical to SEQ ID NO: 15
  Description of Artificial Sequence: Synthetic polypeptide

SEQ ID NO: 173 - protein, 8 aa, Unknown
  Description of Unknown: short peptide sequence
ALNPGSQK

SEQ ID NO: 174 - protein, 10 aa, Unknown
  Description of Unknown: short peptide sequence
GLIAIVMVTI

SEQ ID NO: 175 - protein, 9 aa, Severe acute respiratory syndrome coronavirus 2
FLLPSLATV

SEQ ID NO: 176 - protein, 9 aa, Severe acute respiratory syndrome coronavirus 2
TLGVLVPHV

SEQ ID NO: 177 - protein, 9 aa, Severe acute respiratory syndrome coronavirus 2
YLATALLTL

SEQ ID NO: 178 - protein, 11 aa, Severe acute respiratory syndrome coronavirus 2
VLNEKCSAYTV

SEQ ID NO: 179 - protein, 10 aa, Severe acute respiratory syndrome coronavirus 2
TLACFVLAAV

SEQ ID NO: 180 - protein, 10 aa, Severe acute respiratory syndrome coronavirus 2
FLLPSLATVA

SEQ ID NO: 181 - protein, 11 aa, Severe acute respiratory syndrome coronavirus 2
YVLPNDDTLRV

SEQ ID NO: 182 - protein, 9 aa, Severe acute respiratory syndrome coronavirus 2
YLDAYNMMI

SEQ ID NO: 183 - protein, 11 aa, Severe acute respiratory syndrome coronavirus 2
YLFDESGEFKL

SEQ ID NO: 184 - protein, 9 aa, Unknown
  Description of Unknown: short peptide sequence
ASIINFEKL

SEQ ID NO: 185 - protein, 9 aa, Unknown
  Description of Unknown: short peptide sequence
ESIINFEKL

SEQ ID NO: 186 - protein, 9 aa, Unknown
  Description of Unknown: short peptide sequence
NSLQFFMPV

SEQ ID NO: 187 - protein, 9 aa, Unknown
  Description of Unknown: short peptide sequence
KAMKRYALV

SEQ ID NO: 188 - protein, 9 aa, Unknown
  Description of Unknown: short peptide sequence
ILIPCHRVV

SEQ ID NO: 189 - protein, 11 aa, Unknown
  Description of Unknown: short peptide sequence
IPCHRVVQGDL

SEQ ID NO: 190 - protein, 8 aa, Unknown
  Description of Unknown: short peptide sequence
LNATAIAV

SEQ ID NO: 191 - protein, 10 aa, Unknown
  Description of Unknown: short peptide sequence
NPVPILIPCH

SEQ ID NO: 192 - protein, 9 aa, Unknown
  Description of Unknown: short peptide sequence
ELRSRYWAI

SEQ ID NO: 193 - protein, 8 aa, Unknown
  Description of Unknown: short peptide sequence
RAKFKQLL

SEQ ID NO: 194 - protein, 9 aa, Unknown
  Description of Unknown: short peptide sequence
QAKWRLQTL

SEQ ID NO: 195 - protein, 9 aa, Unknown
  Description of Unknown: short peptide sequence
TAYGERHPW

SEQ ID NO: 196 - protein, 9 aa, Unknown
  Description of Unknown: short peptide sequence
MSFPESQGY

SEQ ID NO: 197 - protein, 9 aa, Unknown
  Description of Unknown: short peptide sequence
RTWKIVTGF

SEQ ID NO: 198 - protein, 8 aa, Unknown
  Description of Unknown: short peptide sequence
NLVPMVAT

SEQ ID NO: 199 - protein, 8 aa, Unknown
  Description of Unknown: short peptide sequence
GLCPIISG

SEQ ID NO: 200 - protein, 8 aa, Unknown
  Description of Unknown: short peptide sequence
GMLNYVDS

SEQ ID NO: 201 - protein, 8 aa, Unknown
  Description of Unknown: short peptide sequence
SLLPAIVE

SEQ ID NO: 202 - protein, 8 aa, Unknown
  Description of Unknown: short peptide sequence
GLLDRLYD

SEQ ID NO: 203 - protein, 8 aa, Unknown
  Description of Unknown: short peptide sequence
WLIGFDFD

SEQ ID NO: 204 - protein, 8 aa, Unknown
  Description of Unknown: short peptide sequence
YMMGIEYG

SEQ ID NO: 205 - protein, 9 aa, Unknown
  Description of Unknown: short peptide sequence
GLCPIISGL

SEQ ID NO: 206 - protein, 9 aa, Unknown
  Description of Unknown: short peptide sequence
GMLNYVDSV

SEQ ID NO: 207 - protein, 9 aa, Severe acute respiratory syndrome coronavirus 2
ALWEIQQVV

SEQ ID NO: 208 - protein, 10 aa, Severe acute respiratory syndrome coronavirus 2
KLPDDFTGCV

SEQ ID NO: 209 - protein, 9 aa, Severe acute respiratory syndrome coronavirus 2
LLYDANYFL

SEQ ID NO: 210 - protein, 12 aa, Severe acute respiratory syndrome coronavirus 2
FGQFCNDPFLGV

SEQ ID NO: 211 - protein, 10 aa, Severe acute respiratory syndrome coronavirus 2
FTYASALWEI

SEQ ID NO: 212 - protein, 11 aa, Unknown
  Description of Unknown: short peptide sequence
ALWEIQQVVDA

SEQ ID NO: 213 - protein, 9 aa, Unknown
  Description of Unknown: short peptide sequence
YQDVNCTEV

SEQ ID NO: 214 - protein, 9 aa, Unknown
  Description of Unknown: short peptide sequence
LLLDRLNQL

SEQ ID NO: 215 - protein, 9 aa, Unknown
  Description of Unknown: short peptide sequence
KIADYNYKL

SEQ ID NO: 216 - protein, 9 aa, Unknown
  Description of Unknown: short peptide sequence
YLQPRTFLL

SEQ ID NO: 217 - protein, 9 aa, Unknown
  Description of Unknown: short peptide sequence
KACPLIAAV

SEQ ID NO: 218 - protein, 11 aa, Unknown
  Description of Unknown: short peptide sequence
KLWAQCVQLHN

SEQ ID NO: 219 - protein, 10 aa, Unknown
  Description of Unknown: short peptide sequence
GLIAIVAVTI

SEQ ID NO: 220 - protein, 9 aa, Unknown
  Description of Unknown: short peptide sequence
KLWAQCVQL

SEQ ID NO: 221 - protein, 9 aa, Unknown
  Description of Unknown: short peptide sequence
NLLKDCPAV

SEQ ID NO: 222 - protein, 9 aa, Unknown
  Description of Unknown: short peptide sequence
VFITLCFTL

SEQ ID NO: 223 - protein, 10 aa, Unknown
  Description of Unknown: short peptide sequence
HFVCNLLLLF

SEQ ID NO: 224 - protein, 9 aa, Unknown
  Description of Unknown: short peptide sequence
TYPVLEEMF

SEQ ID NO: 225 - protein, 9 aa, Unknown
  Description of Unknown: short peptide sequence
QYDPVAALF

SEQ ID NO: 226 - protein, 10 aa, Unknown
  Description of Unknown: short peptide sequence
VYMPASWVMR

SEQ ID NO: 227 - protein, 9 aa, Unknown
  Description of Unknown: short peptide sequence
NYNYLYRLF

SEQ ID NO: 228 - protein, 9 aa, Unknown
  Description of Unknown: short peptide sequence
SYLFQHANL

SEQ ID NO: 229 - protein, 10 aa, Unknown
  Description of Unknown: short peptide sequence
VYYTSNPTTF

SEQ ID NO: 230 - protein, 9 aa, Unknown
  Description of Unknown: short peptide sequence
NYTVSCSPF

SEQ ID NO: 231 - protein, 9 aa, Unknown
  Description of Unknown: short peptide sequence
FYLCFLAFL

SEQ ID NO: 232 - protein, 10 aa, Unknown
  Description of Unknown: short peptide sequence
QFAPSASAFF

SEQ ID NO: 233 - protein, 9 aa, Unknown
  Description of Unknown: short peptide sequence
YFIASFRLF

SEQ ID NO: 234 - protein, 10 aa, Severe acute respiratory syndrome coronavirus 2
GLIAIVMVTI